(19) United States
(12) Patent Application Publication
(10) Pub. No.: US 2011/0053993 A1
(43) Pub. Date: Mar. 3, 2011

(54) PRODRUGS OF DESAZADESFERROTHIOCIN POLYETHER ANALOGUES AS METAL CHELATION AGENTS

(75) Inventors: John M. McCall, JR., Boca Grande, CA (US); Hugh Y. Rienhoff, JR., San Carlos, CA (US)

(73) Assignee: FERROKIN BIOSCIENCES, INC., San Carlos, CA (US)

(21) Appl. No.: 12/843,483

(22) Filed: Jul. 26, 2010

Related U.S. Application Data
(60) Provisional application No. 61/228,690, filed on Jul. 27, 2009.

(51) Int. Cl. A61K 31/426; C07D 277/12; C07D 413/12; A61K 31/427; A61P 7/06; A61P 35/02; A61P 3/10; A61P 1/16; A61P 25/00

(52) U.S. Cl. 514/365; 548/201

(57) ABSTRACT

Disclosed herein are new compounds of desazadesferrothiocin polyether (DADFT-PE) analogues, as well as pharmaceutical compositions comprising them and their application as metal chelation agents for the treatment of disease. Methods of chelation of iron and other metals in a human or animal subject are also provided for the treatment of metal overload and toxicity.

This application claims the benefit of priority of U.S. provisional application No. 61/228,690, filed Jul. 27, 2009, the disclosure of which is incorporated by reference as if written herein in its entirety.

Disclosed herein are prodrugs of desazadesferrothiocin polyether (DADFT-PE) analogues, as well as pharmaceutical compositions comprising them and their application as metal chelation agents for the treatment of disease. Methods of chelation of iron and other metals in a human or animal subject are also provided for the treatment of metal overload and associated toxicity, maldistribution within the body and managing metabolism by therapeutic manipulation of metal levels.

Metal ions are critical to the proper functioning of living systems. Ions such as Fe³⁺, Zn²⁺, Cu²⁺, Ca²⁺, and Co³⁺, to name but a few, can be found in the active sites of over a third of known enzymes and other functional proteins such as RNA polymerase, DNA transcription factors, cytochromes P450s, hemoglobin, myoglobin, and coenzymes such as vitamin B₁₂. There, these metals serve to facilitate oxidation and reduction reactions, stabilize or shield charge distributions, and orient substrates for reactions. Metals are also used as metabolic sensors in conjuction with other molecular entities as part of the biochemical regulation of oxygen, reactive nitrogen species (RNS) such as NO⁻ and reactive oxygen species (ROS), e.g. O₂⁻.

The body, however, has a limited ability to absorb and excrete metals, and an excess can lead to toxicity. As one example, an excess of iron, whether derived from red blood cells chronically transfused, necessary in such conditions such as beta thalassemia major, or from increased absorption of dietary iron such as hereditary hemochromatosis can be toxic through the generation by iron of reactive oxygen species such as H₂O₂. In the presence of Fe²⁺, H₂O₂ is reduced to the hydroxyl radical (HO), a very reactive species, a process known as the Fenton reaction. The hydroxyl radical reacts very quickly with a variety of cellular constituents and can initiate free radicals and radical-mediated chain processes that damage DNA and membranes, as well as produce carcinogens. The clinical result is that without effective treatment, body iron progressively increases with deposition in the liver, heart, pancreas, and elsewhere. Iron accumulation may also produce (i) liver disease that may progress to cirrhosis, (ii) diabetes related both to iron-induced decreases in pancreatic β-cell secretion and increases in hepatic insulin resistance and (iii) heart disease, still the leading cause of death in beta thalassemia major and other anemias associated with transfusional iron overload.

As another example, ions with little or no endogenous function may find their way into the body and effect damage. Heavy metal ions such as Hg²⁺ can replace ions such as Zn²⁺ in metalloproteins and render them inactive, resulting in serious acute or chronic toxicity that can end in a patient's death or in birth defects in that patient's children. Even more significantly, radioactive isotopes of the lanthanide and actinide series can visit grave illness on an individual exposed to them by mouth, air, or skin contact. Such exposure could result not only from the detonation of a nuclear bomb or a “dirty bomb” composed of nuclear waste, but also from the destruction of a nuclear power facility.

Agents for the chelation and decorporation of metal ions in living organisms have been previously disclosed and are in clinical use. A variety of ligands have been shown to bind Fe³⁺, Pu⁴⁺, Th⁴⁺, Am⁴⁺, Eu³⁺ and U⁴⁺, for example. Traditional standard therapies include the use of agents such as deferoxamine (DFO, N′[5-(acetyl-hydroxy-amino)pentyl]-N-[5-[3-(5-aminopentyl-hydroxy-carbamoyl)propanoylamino]pentyl]-N-hydroxy-butane diamide), a very effective metal chelator. DFO is, unfortunately, not orally bioavailable and must therefore be parenterally dosed IV, IP, or SC, and once in the bloodstream has a very short half life. Diethylene triamine pentaacetic acid (DTPA) is approved for use in the treatment of lanthanide and actinide poisoning, but also cannot be dosed orally, ideally should be given very quickly following contamination, and presents with a number of side effects. For these reasons, continuous infusion of these agents is often required, and particularly in the case of chronic disorders, patient compliance is a challenge to achieve the desired therapeutic outcome. A thorough review of publicly available art will show that although effective chelation agents have been available for decades, oral bioavailability has historically been a desirable trait in successive next-generation agents.

More recently, orally active agents have become available for use in the treatment of metal overload. Deferiprone (3-hydroxy-1,2-dimethylpyridin-4(1H)-one) has been used in Europe and some other countries as an oral agent for the treatment of transfusional iron overload in the setting of beta thalassemia and other disorders, but for safety reasons the drug is not approved for use in the United States and Canada except for on a compassionate use basis; reported side effects include life-threatening agranulocytosis which has relegated deferiprone to a second-line therapy. Deferasirox (Exjade, [4-(3Z,5E)-3,5-bis(6-oxo-1-cyclohexa-2,4-dienylidene)-1,2,4-triazolidin-1-yl]benzoic acid, Novartis) is currently the only oral agent approved in the United States for chelation therapy. Notwithstanding, nephrotoxicity leading to renal failure, liver failure and pancytopenia have been reported by the Food and Drug Administration as side effects to deferasirox oral suspension tablets. Moreover, neither of these two agents is as efficacious in chelating iron as DFO. Clearly a clinical need remains in the art for long-lasting, orally active metal chelators with reduced toxicity for the treatment of iron overload secondary to transfusion or excessive intestinal absorption and other metal disorders in which metal levels might be managed for clinical benefit.

Analogues of desferrithiocin, or [(S)-4,5-dihydro-2-(3-hydroxy-2-pyridinyl)4methyl-4-thiazo]carboxylic acid (DFT) have been shown to form 2:1 hexacoordinate complexes with Fe³⁺ and Th⁴⁺. These ligands, when administered either subcutaneously (SC) or orally (PO) to rodents, dogs, and primates, have been shown to clear iron very efficiently, and to decorporate uranium from rodents when given SC, PO, or intraperitoneally, with particularly profound effects in the kidney. Although development of DFT itself had been discontinued due to nephrotoxicity, one of these ligands (S)-2-(2,4-dihydroxyphenyl)-4,5-dihydro-4-methyl-4-thiazolecarboxylic acid, or (S)-4′-(HO)-DADFT, has proven to be an effective chelation agent with the additional benefit of being orally available. A very recent paper discloses the design and testing of DADFT analogues substituted by a polyether group at the 3′, 4′, and 5′ positions (Bergeron R J et al., J Med. Chem. 2007 Jul. 12; 50(14):3302-13). Polyether analogues had uniformly higher iron-clearing efficiencies (ICEs) than their corresponding parent ligands in rodents and in serum albumin binding studies, with the 3′-DADFT-PE analogue (S)-4,5-dihydro-2-[2-hydroxy-3-(3,6,9-trioxadecyloxy)phenyl]-4-methyl-4-thiazolecarboxylic acid showing the most promising ICE in rodents and non-human primates.

Though DADFT polyethers as a class of compounds appear promising in the search for improved metal chelation agents, much work remains to be done in the characterization, development, and selection of a compound suitable for use in humans. Room for improvement is still apparent in the design of analogues which have the optimal balance of bioavailability and other pharmacokinetic parameters, solubility, ICE, target tissue penetration, favorable metabolism and toxicology, and other attributes for the purpose of providing safe and effective compounds which will be easy to use by patients and clinicians alike. Additionally, many factors still influence the suitability of a compound as a pharmaceutical agent in general. For example, to be ideally suited for delivery to patients, compounds should be readily uptaken by the patient's body via the chosen route of administration, should be soluble and bioavailable to the target compartment or organ, and should be cleared from the body in an appropriate period of time. The design of prodrugs presents opportunities for improvements in each of these areas.

Disclosed herein are novel prodrugs of these polyether analogues and derivatives thereof. Pharmaceutical formulations comprising these compounds are also disclosed, as well as methods for the treatment of diseases and conditions related to toxicity which is a result of an acute or chronic excess of metal in a human or animal body.

In certain embodiments, compounds have the structural formula I:

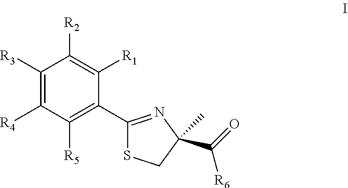

wherein:

R₁, R₂, R₃, R₄, and R₅ are independently chosen from hydrogen, hydroxy, OXR₇, and CH₃O((CH₂)ₙ—O)ₘ—, any of which may be optionally substituted;

m is an integer from 0 to 8;

n is an integer from 0 to 8;

R₆ is chosen from OR₈ and SR₉,

R₇ is chosen from hydrogen, NR₁₀R₁₁, lower alkyl, aralkyl, and aryl, any of which may be optionally substituted;

R₈ is chosen from hydrogen, C₄-C₈ alkyl, and lower aralkyl;

R₉ is chosen from hydrogen, lower alkyl, and lower aralkyl;

R₁₀ and R₁₁ are each independently chosen from hydrogen, lower alkyl, and aryl, any of which may be optionally substituted, or R₁₀ and R₁₁ taken together may form a heterocycloalkyl or heteroaryl; and

X is chosen from a bond and C(O);

wherein at least one of R₁-R₅ is CH₃O((CH₂)ₙ—O)ₘ—;

at least one of R₁-R₅ is optionally substituted OXR₇; and

R₇, R₈, and R₉ can not all be hydrogen.

Certain compounds and prodrugs disclosed herein may possess useful metal chelating activity, and may be used in the treatment or prophylaxis of a disease or condition in which metal excess, toxicity, or maldistribution plays a contributing or active role. Thus, in broad aspect, certain embodiments also provide pharmaceutical compositions comprising one or more compound or prodrug disclosed herein together with a pharmaceutically acceptable carrier, as well as methods of making and using the compounds and prodrugs and their compositions. Certain embodiments provide methods for chelating metals in living systems. Other embodiments provide methods for treating disorders and symptoms relating to metal toxicity in a patient in need of such treatment, comprising administering to said patient a therapeutically effective amount of a compound or composition according to the present invention, or a prodrug thereof. Also provided is the use of certain compounds and prodrugs disclosed herein for use in the manufacture of a medicament for the treatment of a disease or condition ameliorated by the chelation or decorporation of metals.

In certain embodiments, compounds have structural formula II:

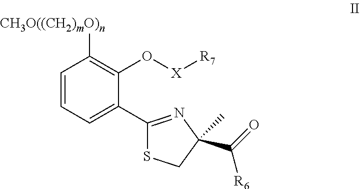

wherein:

m is an integer from 0 to 8;

n is an integer from 0 to 8;

R₆ is chosen from OR₈ and SR₉;

R₇ is chosen from hydrogen, NR₁₀R₁₁, lower alkyl, lower aralkyl, and lower aryl, any of which may be optionally substituted;

R₈ is chosen from hydrogen, C₄-C₈ alkyl, and lower aralkyl;

R₉ is chosen from hydrogen, lower alkyl, and lower aralkyl;

R₁₀ and R₁₁ are each independently chosen from hydrogen, lower alkyl, and aryl, any of which may be optionally substituted, or R₁₀ and R₁₁ taken together may form a lower heterocycloalkyl or heteroaryl; and

X is chosen from a bond and C(O);

wherein at least one of R₁-R₅ is CH₃O((CH₂)ₙ—O)ₘ—; and

R₇, R₈, and R₉ can not all be hydrogen.

In further embodiments compounds have the structural formula II wherein:

m is 2; and

n is 3.

In further embodiments compounds have the structural formula II wherein:

X is C(O); and

R₇ is chosen from NR₁₀R₁₁, lower alkyl, lower aralkyl, and lower aryl, any of which may be optionally substituted.

In yet further embodiments compounds have the structural formula II wherein:

R₇ is NR₁₀R₁₁; and

R₁₀ and R₁₁ taken together form a lower heterocycloalkyl.

In another embodiment compounds have the structural formula II, wherein R₁₀ and R₁₁ taken together form pyrrolidine, piperidine, morpholine, azepine, diazepine, piperazine, or azetidine.

In another embodiment compounds have the structural formula II, wherein:

R₈ is chosen from hydrogen, C₄-C₈ alkyl, and aralkyl;

and R₉ is chosen from hydrogen, lower alkyl and lower aralkyl.

In a further embodiment compounds have the structural formula II, wherein:

R₈ is isobutyl; and

R₉ is chosen from ethyl and isobutyl.

In yet another embodiment compounds have the structural formula II, wherein

X is a bond;

R₇ is hydrogen; and

R₈ is chosen from C₄-C₈ alkyl, and lower aralkyl;

and R₉ is chosen from lower alkyl and lower aralkyl.

In yet another embodiment compounds have the structural formula II, wherein

X is a bond;

R₇ is hydrogen;

R₈ is isobutyl; and

R₉ is chosen from ethyl and isobutyl.

In further embodiments, compounds have structural formula III:

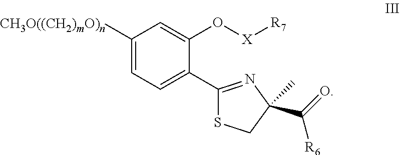

wherein:

m is an integer from 0 to 8;

n is an integer from 0 to 8;

R₆ is chosen from OR₈ and SR₉,

R₇ is chosen from hydrogen, NR₁₀R₁₁, lower alkyl, lower aralkyl, and lower aryl, any of which may be optionally substituted;

R₈ is chosen from hydrogen, C₄-C₈ alkyl, and lower aralkyl;

R₉ is chosen from hydrogen, lower alkyl, and lower aralkyl;

R₁₀ and R₁₁ are each independently chosen from hydrogen, lower alkyl, and aryl, any of which may be optionally substituted, or R₁₀ and R₁₁ taken together may form a lower heterocycloalkyl or heteroaryl; and

X is chosen from a bond and C(O);

wherein at least one of R₁-R₅ is CH₃O((CH₂)ₙ—O)ₘ—; and

R₇, R₈, and R₉ can not all be hydrogen.

In further embodiments compounds have the structural formula III wherein:

m is 2; and

n is 3.

In further embodiments compounds have the structural formula III wherein:
X is C(O); and
R₇ is chosen from NR₁₀R₁₁, lower alkyl, lower aralkyl, and lower aryl, any of which may be optionally substituted.

In yet further embodiments compounds have the structural formula III wherein:

R₇ is NR₁₀R₁₁; and

R₁₀ and R₁₁ taken together form a lower heterocycloalkyl.

In another embodiment compounds have the structural formula III, wherein R₁₀ and R₁₁ taken together form pyrrolidine, piperidine, morpholine, azepine, diazepine, piperazine, or azetidine.

In another embodiment compounds have the structural formula III, wherein:

R₈ is chosen from hydrogen, C₄-C₈ alkyl, and lower aralkyl; and

R₉ is chosen from hydrogen, lower alkyl and lower aralkyl.

In a further embodiment compounds have the structural formula III, wherein:

R₈ is isobutyl; and

R₉ is chosen from ethyl and isobutyl.

In yet another embodiment compounds have the structural formula III, wherein:

X is a bond;

R₇ is hydrogen; and

R₈ is chosen from C₄-C₈ alkyl and lower aralkyl; and

R₉ is chosen from lower alkyl and lower aralkyl.

In yet another embodiment compounds have the structural formula III, wherein:

X is a bond;

R₇ is hydrogen;

R₈ is isobutyl; and

R₉ is chosen from ethyl and isobutyl.

In further embodiments, compounds have structural formula IV:

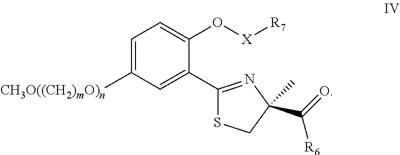

wherein:

m is an integer from 0 to 8;

n is an integer from 0 to 8;

R₆ is chosen from OR₈ and SR₉;

R₇ is chosen from hydrogen, NR₁₀R₁₁, lower alkyl, lower aralkyl, and lower aryl, any of which may be optionally substituted;

R₈ is chosen from hydrogen, C₄-C₈ alkyl, and lower aralkyl;

R₉ is chosen from hydrogen, alkyl, and aralkyl;

R₁₀ and R₁₁ are each independently chosen from hydrogen, lower alkyl, and aryl, any of which may be optionally substituted, or R₁₀ and R₁₁ taken together may form a lower heterocycloalkyl or heteroaryl; and

X is chosen from a bond and C(O);

wherein at least one of R₁-R₅ is CH₃O((CH₂)ₙ—O)ₘ—; and

R₇, R₈, and R₉ can not all be hydrogen.

In further embodiments compounds have the structural formula IV wherein:

m is 2; and

n is 3.

In further embodiments compounds have the structural formula IV wherein:
X is C(O); and
R₇ is chosen from NR₁₀R₁₁, lower alkyl, lower aralkyl, and lower aryl, any of which may be optionally substituted.

In yet further embodiments compounds have the structural formula IV wherein:

R₇ is NR₁₀R₁₁; and

R₁₀ and R₁₁ taken together form a lower heterocycloalkyl.

In another embodiment compounds have the structural formula IV, wherein R₁₀ and R₁₁ taken together form pyrrolidine, piperidine, morpholine, azepine, diazepine, piperazine, or azetidine.

In another embodiment compounds have the structural formula IV, wherein:
R₈ is chosen from hydrogen, C₄-C₈ alkyl, and lower aralkyl; and
R₉ are each independently chosen from hydrogen, lower alkyl and lower aralkyl.

In a further embodiment compounds have the structural formula IV, wherein R₈ is isobutyl, and

R₉ is chosen from ethyl and isobutyl.

In yet another embodiment compounds have the structural formula IV, wherein

X is a bond;

R₇ is hydrogen;

R₈ is chosen from C₄-C₈ alkyl and lower aralkyl; and

R₉ is chosen from lower alkyl and lower aralkyl.

In yet another embodiment compounds have the structural formula IV, wherein

X is a bond;

R₇ is hydrogen; and

R₈ is isobutyl; and

R₉ is chosen from ethyl and isobutyl.

In further embodiments, compounds have structural formula V:

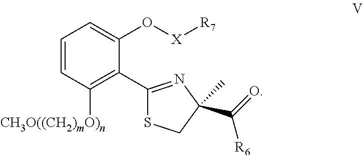

wherein:

m is an integer from 0 to 8;

n is an integer from 0 to 8;

R₆ is chosen from OR₈ and SR₉;

R₇ is chosen from hydrogen, NR₁₀R₁₁, lower alkyl, lower aralkyl, and lower aryl, any of which may be optionally substituted;

R₈ is chosen from hydrogen, C₄-C₈ alkyl, and lower aralkyl;

R₉ is chosen from hydrogen, lower alkyl, and lower aralkyl;

R₁₀ and R₁₁ are each independently chosen from hydrogen, lower alkyl, and aryl, any of which may be optionally substituted, or R₁₀ and R₁₁ taken together may form a lower heterocycloalkyl or heteroaryl; and

X is chosen from a bond and C(O);

wherein at least one of R₁-R₅ is CH₃O((CH₂)ₙ—O)ₘ—; and

R₇, R₈, and R₉ can not all be hydrogen.

In further embodiments compounds have the structural formula V wherein:

m is 2; and

n is 3.

In further embodiments compounds have the structural formula V wherein:
X is C(O); and
R₇ is chosen from NR₁₀R₁₁, lower alkyl, lower aralkyl, and lower aryl, any of which may be optionally substituted.

In yet further embodiments compounds have the structural formula V wherein:

R₇ is NR₁₀R₁₁; and

R₁₀ and R₁₁ taken together form a lower heterocycloalkyl.

In another embodiment compounds have the structural formula IV, wherein R₁₀ and R₁₁ taken together form pyrrolidine, piperidine, morpholine, azepine, diazepine, piperazine, or azetidine.

In another embodiment compounds have the structural formula V, wherein:

R₈ is chosen from hydrogen, C₄-C₈ alkyl, and lower aralkyl; and

R₉ is chosen from hydrogen, lower alkyl and lower aralkyl.

In a further embodiment compounds have the structural formula V, wherein:

R₈ is isobutyl, and

R₉ is chosen from ethyl and isobutyl.

In yet another embodiment compounds have the structural formula V, wherein:

X is a bond;

R₇ is hydrogen; and

R₈ is chosen from C₄-C₈ alkyl and lower aralkyl; and

R₉ is chosen from lower alkyl and lower aralkyl.

In yet another embodiment compounds have the structural formula V, wherein

X is a bond;

R₇ is hydrogen;

R₈ is isobutyl; and

R₉ is chosen from ethyl and isobutyl.

In further embodiments, compounds have structural formula VI:

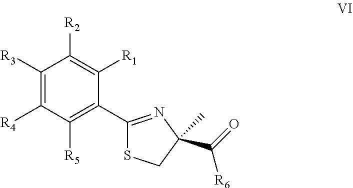

wherein:

R₁, R₂, R₃, R₄, and R₅ are independently chosen from hydrogen, hydroxy, OXR₇, and CH₃O((CH₂)ₙ—O)ₘ—, any of which may be optionally substituted;

m is an integer from 0 to 8;

n is an integer from 0 to 8;

R₆ is chosen from OR₈ and SR₉,

R₇ is chosen from hydrogen, NR₁₀R₁₁, lower alkyl, aralkyl, and aryl, any of which may be optionally substituted;

R₈ is chosen from C₄-C₈ alkyl and lower aralkyl;

R₉ is chosen from hydrogen, lower alkyl, and lower aralkyl;

R₁₀ and R₁₁ are each independently chosen from hydrogen, lower alkyl, and aryl, any of which may be optionally substituted, or R₁₀ and R₁₁ taken together may form a heterocycloalkyl or heteroaryl; and

X is chosen from a bond and C(O);

wherein at least one of R₁-R₅ is CH₃O((CH₂)ₙ—O)ₘ—;

at least one of R₁-R₅ is optionally substituted OXR₇.

In further embodiments, compounds have structural formula VII:

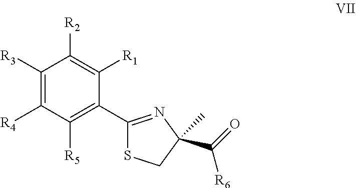

wherein:

R₁, R₂, R₃, R₄, and R₅ are independently chosen from hydrogen, hydroxy, OXR₇, and CH₃O((CH₂)ₙ—O)ₘ—, any of which may be optionally substituted;

m is an integer from 0 to 8;

n is an integer from 0 to 8;

R₆ is chosen from OR₈ and SR₉;

R₇ is chosen from NR₁₀R₁₁, lower alkyl, aralkyl, and aryl, any of which may be optionally substituted;

R₈ is chosen from hydrogen, C₄-C₈ alkyl, and lower aralkyl;

R₉ is chosen from hydrogen, lower alkyl, and lower aralkyl;

R₁₀ and R₁₁ are each independently chosen from hydrogen, lower alkyl, and aryl, any of which may be optionally substituted, or R₁₀ and R₁₁ taken together may form a heterocycloalkyl or heteroaryl; and

X is chosen from a bond and C(O);

wherein at least one of R₁-R₅ is CH₃O((CH₂)ₙ—O)ₘ—;

at least one of R₁-R₅ is optionally substituted OXR₇.

In further embodiments compounds have the structural formula VII, wherein:

R₈ is chosen from C₄-C₈ alkyl and lower aralkyl.

In certain embodiments of the present invention are provided pharmaceutical compositions comprising the prodrug as disclosed herein together with at least one pharmaceutically acceptable excipient.

In certain embodiments of the present invention are provided a method of treating a metal-mediated condition in a subject comprising administering to the subject a therapeutically effective amount of a compound therapeutically effective amount of a compound of formula I.

In another embodiment, said metal is trivalent

In further embodiments, said condition is responsive to the chelation, sequestration, or elimination of metal.

In further embodiments, said metal is iron.

In further embodiments, said condition is iron overload.

In further embodiments, said condition is the result of mal-distribution or redistribution of iron in the body.

In further embodiments, said condition is chosen from atransferrinemia, aceruloplasminemia, and Fredreich's ataxia.

In further embodiments, said condition is the result of transfusional iron overload.

In further embodiments, said condition is chosen from beta-thalassemia major and intermedia, sickle cell anemia, Diamond-Blackfan anemia, sideroblastic anemia, chronic hemolytic anemias, off-therapy leukemias, bone marrow transplant and myelodysplastic syndrome.

In further embodiments, said condition is a hereditary condition resulting in the excess absorption of dietary iron.

In further embodiments, said condition is chosen from hereditary hemochromatosis and porphyria cutanea tarda.

In further embodiments, said condition is diabetes.

In further embodiments, said condition is an acquired disease that results in excess dietary iron absorption.

In further embodiments, said condition is a liver disease.

In further embodiments, said disease is hepatitis.

In further embodiments, said metal is a lanthanide or actinide.

In further embodiments, said pathological condition is lanthanide or actinide overload.

In further embodiments, the therapeutically effective amount of a compound as disclosed herein that induces the bodily excretion of iron or other trivalent metal is greater than 0.2 mg/kg/d in the subject.

In further embodiments, the therapeutically effective amount of a compound as disclosed herein can be given at a dose of at least 10 mg/kg/d without clinically apparent toxic effects on the kidney, bone marrow, thymus, liver, spleen, heart or adrenal glands.

As used herein, the terms below have the meanings indicated.

When ranges of values are disclosed, and the notation “from n₁ . . . to n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon group having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(—CH═CH—), (—C::C—)]. Examples of suitable alkenyl groups include ethenyl, propenyl, 2-methylpropenyl, 1,4-butadienyl and the like. Unless otherwise specified, the term “alkenyl” may include “alkenylene” groups.

The term “alkoxy,” as used herein, alone or in combination, refers to an alkyl ether group, wherein the term alkyl is as defined below. Examples of suitable alkyl ether groups include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl group containing from 1 to 20 carbon atoms. In certain embodiments, said alkyl will comprise from 1 to 10 carbon atoms. In further embodiments, said alkyl will comprise from 1 to 6 carbon atoms. Alkyl groups may be optionally substituted as defined herein. Examples of alkyl groups include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkynyl,” as used herein, alone or in combination, refers to a straight-chain or branched chain hydrocarbon group having one or more triple bonds and containing from 2 to 20 carbon atoms. In certain embodiments, said alkynyl comprises from 2 to 6 carbon atoms. In further embodiments, said alkynyl comprises from 2 to 4 carbon atoms. The term “alkynylene” refers to a carbon-carbon triple bond attached at two positions such as ethynylene (—C:::C—, —C≡C—). Examples of alkynyl groups include ethynyl, propynyl, hydroxypropynyl, butyn-1-yl, butyn-2-yl, pentyn-1-yl, 3-methylbutyn-1-yl, hexyn-2-yl, and the like. Unless otherwise specified, the term “alkynyl” may include “alkynylene” groups.

The terms “amido” and “carbamoyl,” as used herein, alone or in combination, refer to an amino group as described below attached to the parent molecular moiety through a carbonyl group, or vice versa. The term “C-amido” as used herein, alone or in combination, refers to a —C(═O)—NR₂ group with R as defined herein. The term “N-amido” as used herein, alone or in combination, refers to a RC(═O)NH— group, with R as defined herein. The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to —NRR′, wherein R and R′ are independently chosen from hydrogen, alkyl, acyl, heteroalkyl, aryl, cycloalkyl, heteroaryl, and heterocycloalkyl, any of which may themselves be optionally substituted. Additionally, R and R′ may combine to form heterocycloalkyl, either of which may be optionally substituted.

The term “aryl,” as used herein, alone or in combination, means a carbocyclic aromatic system containing one, two or three rings wherein such polycyclic ring systems are fused together. The term “aryl” embraces aromatic groups such as phenyl, naphthyl, anthracenyl, and phenanthryl.

The terms “benzo” and “benz,” as used herein, alone or in combination, refer to the divalent group C₆H₄═ derived from benzene. Examples include benzothiophene and benzimidazole.

The term “carbonyl,” as used herein, when alone includes formyl [—C(O)H] and in combination is a —C(O)— group.

The term “carboxyl” or “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl group wherein each cyclic moiety contains from 3 to 12 carbon atom ring members and which may optionally be a benzo fused ring system which is optionally substituted as defined herein. In certain embodiments, said cycloalkyl will comprise from 5 to 7 carbon atoms. Examples of such cycloalkyl groups include cyclopropyl, cyclobutyl, cyclopentyl, cyclohexyl, cycloheptyl, tetrahydronapthyl, indanyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl group having the meaning as defined above wherein one or more hydrogens are replaced with a halogen. Specifically embraced are monohaloalkyl, dihaloalkyl and polyhaloalkyl groups. A monohaloalkyl group, for one example, may have an iodo, bromo, chloro or fluoro atom within the group. Dihalo and polyhaloalkyl groups may have two or more of the same halo atoms or a combination of different halo groups. Examples of haloalkyl groups include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or cyclic hydrocarbon group, or combinations thereof, fully saturated or containing from 1 to 3 degrees of unsaturation, consisting of the stated number of carbon atoms and from one to three heteroatoms chosen from O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH₂—NH—OCH₃.

The term “heteroaryl,” as used herein, alone or in combination, refers to a 3 to 7 membered unsaturated heteromonocyclic ring, or a fused monocyclic, bicyclic, or tricyclic ring system in which at least one of the fused rings is aromatic, which contains at least one atom chosen from O, S, and N. In certain embodiments, said heteroaryl will comprise from 5 to 7 carbon atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated monocyclic, bicyclic, or tricyclic heterocyclic group containing at least one heteroatom as a ring member, wherein each said heteroatom may be independently chosen from nitrogen, oxygen, and sulfur. In certain embodiments, said heterocycloalkyl will comprise from 1 to 4 heteroatoms as ring members. In further embodiments, said heterocycloalkyl will comprise from 1 to 2 heteroatoms as ring members. In certain embodiments, said heterocycloalkyl will comprise from 3 to 8 ring members in each ring. In further embodiments, said heterocycloalkyl will comprise from 3 to 7 ring members in each ring. In yet further embodiments, said heterocycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihy-dropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The phrase “in the main chain” refers to the longest contiguous or adjacent chain of carbon atoms starting at the point of attachment of a group to the compounds of any one of the formulas disclosed herein.

The term “lower,” as used herein, alone or in a combination, where not otherwise specifically defined, means containing from 1 to and including 6 carbon atoms.

The terms “oxy” or “oxa,” as used herein, alone or in combination, refer to —O—.

The term “oxo,” as used herein, alone or in combination, refers to —O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, ester, acyl, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety chosen from hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(O)N(R)— may be attached to the parent moiety at either the carbon or the nitrogen.

Asymmetric centers exist in the compounds disclosed herein. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The phrase “therapeutically effective” is intended to qualify the amount of active ingredients used in the treatment of a disease or disorder. This amount will achieve the goal of reducing or eliminating the said disease or disorder.

The term “chelation” as used herein means to coordinate (as in a metal ion) with and inactivate. Chelation also includes decorporation, a term which itself encompasses chelation and excretion.

The term “iron-clearing efficiency (ICE)” as used herein refers to the efficaciousness of a given concentration of chelator in clearing iron from the body or one of its organs or parts. Efficaciousness in turn concerns quantity of iron removed from a target system (which may be a whole body, an organ, or other) in a unit of time. Chelators are needed for three clinical situations: for acute iron toxicity from ingestion or infusion of iron; to reduce total body iron secondary to transfusion or excess iron absorption; for maintenance of iron balance after total body iron has been satisfactorally reduces and only daily dietary iron needs to be excreted. In practical terms, therefore, for chronic iron overload secondary to transfusion, the recommendation is that between 0.3 and 0.5 mg Fe/kg body weight of the patient per day need be excreted. For the maintenance treatment, 0.25-1 mg/kg/d is sufficient.

The term “therapeutically acceptable” refers to those compounds (or salts, polymorphs, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein, reference to “treatment” of a patient is intended to include prophylaxis. The term “patient” means all mammals including humans. Examples of patients include humans, cows, dogs, cats, goats, sheep, pigs, and rabbits. Preferably, the patient is a human.

The term “prodrug” refers to a compound that is made more active in vivo. Certain compounds disclosed herein may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

The compounds disclosed herein can exist as therapeutically acceptable salts. The present invention includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds disclosed herein, and the like.

Basic addition salts can be prepared during the final isolation and purification of the compounds, often by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium (e.g., NaOH), potassium (e.g., KOH), calcium (including Ca(OH)₂), magnesium (including Mg(OH)₂ and magnesium acetate), zinc, (including Zn(OH)₂ and zinc acetate) and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N-dibenzylethylenediamine. Other representative organic amines useful for the formation of base addition salts include ethylenediamine, ethanolamine, diethanolamine, piperidine, piperazine, choline hydroxide, hydroxyethyl morpholine, hydroxyethyl pyrrolidone, imidazole, n-methyl-d-glucamine, N,N′-dibenzylethylenediamine, N,N′-diethylethanolamine, N,N′-dimethylethanolamine, triethanolamine, and tromethamine. Basic amino acids such as 1-glycine and 1-arginine, and amino acids which may be zwitterionic at neutral pH, such as betaine (N,N,N-trimethylglycine) are also contemplated.

In certain embodiments, the salts may include calcium, magnesium, potassium, sodium, zinc, and piperazine salts of compounds disclosed herein.

Salts disclosed herein may combine in 1:1 molar ratios, and in fact this is often how they are initially synthesized. However, it will be recognized by one of skill in the art that the stoichiometry of one ion in a salt to the other may be otherwise. Salts shown herein may be, for the sake of convenience in notation, shown in a 1:1 ratio; all possible stoichiometric arrangements are encompassed byt the scope of the present invention.

The terms, “polymorphs” and “polymorphic forms” and related terms herein refer to crystal forms of the same molecule, and different polymorphs may have different physical properties such as, for example, melting temperatures, heats of fusion, solubilities, dissolution rates and/or vibrational spectra as a result of the arrangement or conformation of the molecules in the crystal lattice. The differences in physical properties exhibited by polymorphs affect pharmaceutical parameters such as storage stability, compressibility and density (important in formulation and product manufacturing), and dissolution rates (an important factor in bioavailability). Differences in stability can result from changes in chemical reactivity (e.g. differential oxidation, such that a dosage form discolors more rapidly when comprised of one polymorph than when comprised of another polymorph) or mechanical changes (e.g. tablets crumble on storage as a kinetically favored polymorph converts to thermodynamically more stable polymorph) or both (e.g., tablets of one polymorph are more susceptible to breakdown at high humidity). As a result of solubility/dissolution differences, in the extreme case, some polymorphic transitions may result in lack of potency or, at the other extreme, toxicity. In addition, the physical properties of the crystal may be important in processing, for example, one polymorph might be more likely to form solvates or might be difficult to filter and wash free of impurities (i.e., particle shape and size distribution might be different between polymorphs).

Polymorphs of a molecule can be obtained by a number of methods, as known in the art. Such methods include, but are not limited to, melt recrystallization, melt cooling, solvent recrystallization, desolvation, rapid evaporation, rapid cooling, slow cooling, vapor diffusion and sublimation.

Techniques for characterizing polymorphs include, but are not limited to, differential scanning calorimetry (DSC), X-ray powder diffractometry (XRPD), single crystal X-ray diffractometry, vibrational spectroscopy, e.g. IR and Raman spectroscopy, solid state NMR, hot stage optical microscopy, scanning electron microscopy (SEM), electron crystallography and quantitative analysis, particle size analysis (PSA), surface area analysis, solubility studies and dissolution studies.

The term, “solvate,” as used herein, refers to a crystal form of a substance which contains solvent. The term “hydrate” refers to a solvate wherein the solvent is water.

The term, “desolvated solvate,” as used herein, refers to a crystal form of a substance which can only be made by removing the solvent from a solvate.

The term “amorphous form,” as used herein, refers to a noncrystalline form of a substance.

The term “solubility” is generally intended to be synonymous with the term “aqueous solubility,” and refers to the ability, and the degree of the ability, of a compound to dissolve in water or an aqueous solvent or buffer, as might be found under physiological conditions. Aqueous solubility is, in and of itself, a useful quantitative measure, but it has additional utility as a correlate and predictor, with some limitations which will be clear to those of skill in the art, of oral bioavailability. In practice, a soluble compound is generally desirable, and the more soluble, the better. There are notable exceptions; for example, certain compounds intended to be administered as depot injections, if stable over time, may actually benefit from low solubility, as this may assist in slow release from the injection site into the plasma. Solubility is typically reported in mg/mL, but other measures, such as g/g, may be used. Solubilities typically deemed acceptable may range from 1 mg/mL into the hundreds or thousands of mg/mL.

While it may be possible for the compounds and prodrugs disclosed herein to be administered as the raw chemical, it is also possible to present them as a pharmaceutical formulation. Accordingly, provided herein are pharmaceutical formulations which comprise one or more of certain compounds and prodrugs disclosed herein, or one or more pharmaceutically acceptable salts, esters, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, intranasal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound of the subject invention or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the compounds and prodrugs disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds and prodrugs may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds prodrugs may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds and prodrugs which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds and prodrugs to allow for the preparation of highly concentrated solutions.

In addition to the formulations described previously, a compound or prodrug as disclosed herein may also be formulated as a depot preparation. Such long acting formulations may be administered by implantation (for example subcutaneously or intramuscularly) or by intramuscular injection. Thus, for example, the compounds and prodrugs may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds and prodrugs may also be formulated in rectal compositions such as suppositories or retention enemas, e.g., containing conventional suppository bases such as cocoa butter, polyethylene glycol, or other glycerides.

Certain compounds and prodrugs disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

For administration by inhalation, compounds and prodrugs may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds and prodrugs disclosed herein may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Intranasal delivery, in particular, may be useful for delivering compounds to the CNS. It had been shown that intranasal drug administration is a noninvasive method of bypassing the blood-brain barrier (BBB) to deliver neurotrophins and other therapeutic agents to the brain and spinal cord. Delivery from the nose to the CNS occurs within minutes along both the olfactory and trigeminal neural pathways. Intranasal delivery occurs by an extracellular route and does not require that drugs bind to any receptor or undergo axonal transport. Intranasal delivery also targets the nasal associated lymphatic tissues (NALT) and deep cervical lymph nodes. In addition, intranasally administered therapeutics are observed at high levels in the blood vessel walls and perivascular spaces of the cerebrovasculature. Using this intranasal method in animal models, researchers have successfully reduced stroke damage, reversed Alzheimer's neurodegeneration, reduced anxiety, improved memory, stimulated cerebral neurogenesis, and treated brain tumors. In humans, intranasal insulin has been shown to improve memory in normal adults and patients with Alzheimer's disease. Hanson L R and Frey W H, 2^{nd}, J Neuroimmune Pharmacol. 2007 March; 2(1):81-6. Epub 2006 Sep. 15.

Preferred unit dosage formulations are those containing an effective dose, as herein below recited, or an appropriate fraction thereof, of the active ingredient.

It should be understood that in addition to the ingredients particularly mentioned above, the formulations described above may include other agents conventional in the art having regard to the type of formulation in question, for example those suitable for oral administration may include flavoring agents.

Compounds and prodrugs may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compound or prodrug which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds and prodrugs can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds and prodrugs described herein (or a pharmaceutically acceptable salt or ester thereof) in combination with another therapeutic agent. By way of example only, if one of the side effects experienced by a patient upon receiving one of the compounds herein for the treatment of actinide poisoning is depletion of essential trace minerals required by the body for proper functioning, then it may be appropriate to administer a strong chelating agent in combination with supplements of essential trace minerals required by the body for proper functioning, for example zinc and magnesium, to replace those which will inadvertently be lost to chelation therapy. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for thalassemia involving administration of one of the compounds described herein, increased therapeutic benefit may result by also providing the patient with another therapeutic agent for thalassemis, for example deferoxamine. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of certain compounds of the invention with: deferasirox, deferiprone, deferoxamine, DTPA (diethylene triamine pentaacetic acid), EGTA (ethylene glycol tetraacetic acid), EDTA (ethylenediamine tetraacetic acid), DMSA (dimercaptosuccinic acid), DMPS (dimercapto-propane sulfonate), BAL (dimercaprol), BAPTA (aminophenoxyethane-tetraacetic acid), D-penicillamine, and alpha lipoic acid.

In any case, the multiple therapeutic agents (at least one of which is a compound disclosed herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, certain embodiments provide methods for treating disorders and symptoms relating to metal toxicity in a human or animal subject in need of such treatment comprising administering to said subject an amount of a compound disclosed herein effective to reduce or prevent said disorder in the subject, in combination with at least one additional agent for the treatment of said disorder that is known in the art. In a related aspect, certain embodiments provide therapeutic compositions comprising at least one compound disclosed herein in combination with one or more additional agents for the treatment of disorders and symptoms relating to metal toxicity.

Specific diseases to be treated by the compounds, compositions, and methods disclosed herein include iron overload or mal-distribution or redistribution of iron in the body such as atransferrinemia, aceruloplasminemia, or Fredreich's ataxia; transfusional iron overload such as with beta-thalassemia major and intermedia, sickle cell anemia, Diamond-Blackfan anemia, sideroblastic anemia, chronic hemolytic anemias, off-therapy leukemias, bone marrow transplant or myelodysplastic syndrome; a hereditary condition resulting in the excess absorption of dietary iron such as hereditary hemochromatosis, or porphyria cutanea tarda; an acquired disease that results in excess dietary iron absorption such as hepatitis; and other liver diseases; lanthanide or actinide acute poisoning or chronic overload.

Besides being useful for human treatment, certain compounds and formulations disclosed herein may also be useful for veterinary treatment of companion animals, exotic animals and farm animals, including mammals, rodents, and the like. More preferred animals include horses, dogs, and cats.

All references, patents or applications, U.S. or foreign, cited in the application are hereby incorporated by reference as if written herein in their entireties. Where any inconsistencies arise, material literally disclosed herein controls.

### General Synthetic Methods for Preparing Compounds

Certain compounds from which prodrugs of the invention may be formed can be synthesized as described in Bergeron, R J et al., “Design, Synthesis, and Testing of Non-Nephrotoxic Desazadesferrithiocin Polyether Analogues,” J Med. Chem. 2008, 51(13), 3913-23.

The following scheme can generally be used to practice the present invention.

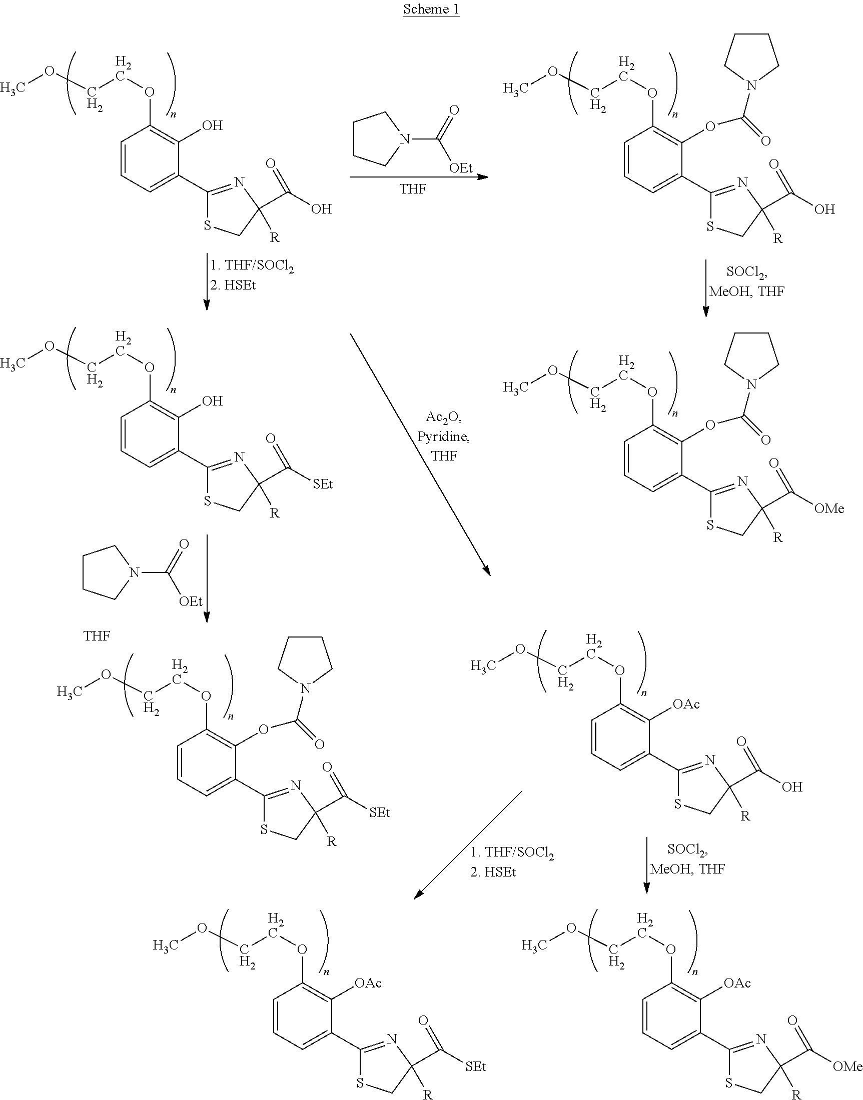

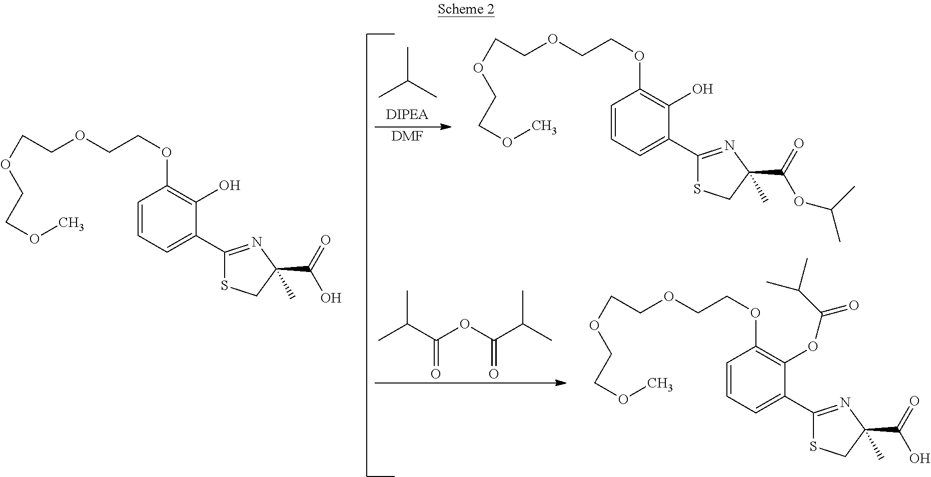

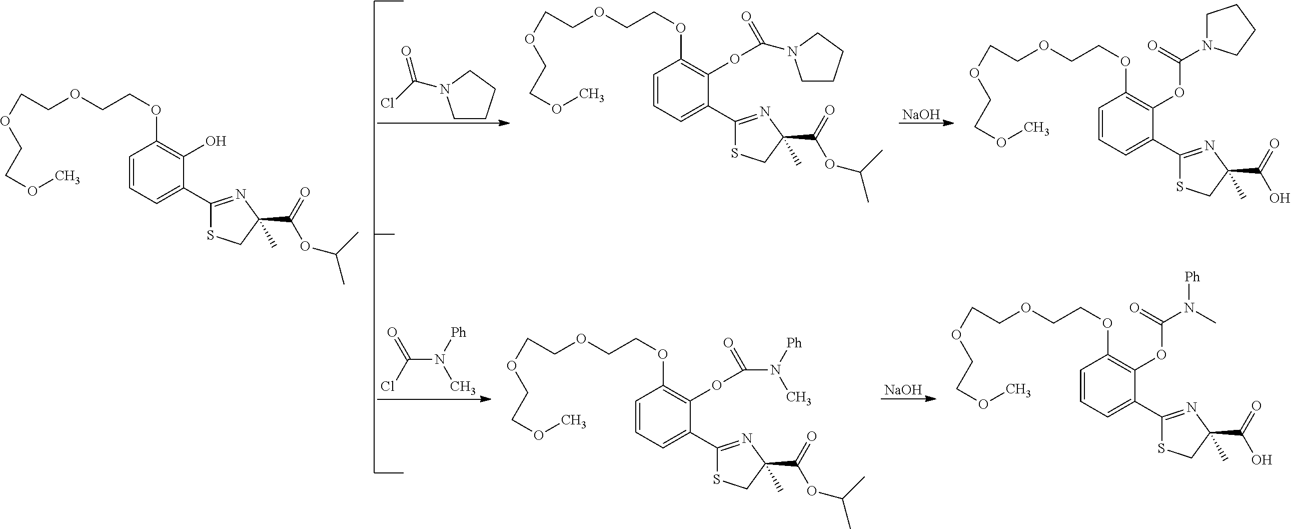

The invention is further illustrated by the following examples.

### EXAMPLE 1

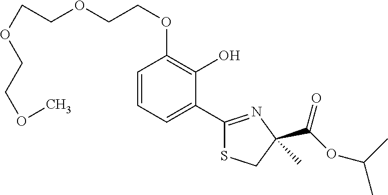

Into a 50-mL 3-necked round-bottom flask purged and maintained with an inert atmosphere of nitrogen, was placed a solution of (S)-2-(2-hydroxy-3-(2-(2-(2-methoxyethoxy)ethoxy)ethoxy)phenyl)-4-methyl-4,5-dihydrothiazole-4-carboxylic acid (1 g, 2.51 mmol, 1.00 equiv) in N,N-dimethylformamide (10 mL), 2-iodopropane (810 mg, 4.76 mmol, 1.90 equiv), N,N-Diisopropylethylamine (614 mg, 4.73 mmol, 1.90 equiv). The resulting solution was stirred for 7 days at room temperature in an oil bath. The resulting mixture was concentrated under vacuum. The crude product (1 g) was purified by Prep-HPLC (AGILENT Pre-HPLC; Column: SunFire Prep C18, 5 um, 19*100 mm; mobile phase, 0.05% TFA aqueous solution and CH₃CN (50% CH3CN up to 70% in 6 min, up to 100% in 0.1 min, hold 100% in 0.9 min; Detector, UV 254 & 220 nm) to yield 300 mg (27%) of (S)-isopropyl 2-(2-hydroxy-3-(2-(2-(2-methoxyethoxy)ethoxy)ethoxy)phenyl)-4-methyl-4,5-dihydrothiazole-4-carboxylate as yellow oil. LC-MS: (ES, m/z): 442 [M+H]⁺. ¹HNMR (DMSO-d₆, 300 MHz, ppm): δ12.58 (br, 1H), 7.18 (t, J=0.9 Hz, 1H), 7.05 (t, J=0.9 Hz, 1H), 6.86 (t, J=7.8 Hz, 1H), 4.98 (m, 1H), 4.12 (m, 2H), 3.76 (m, 3H), 3.61˜3.35 (m, 9H), 3.22 (s, 3H), 1.58 (s, 3H), 1.24 (m, 6H).

### EXAMPLE 2

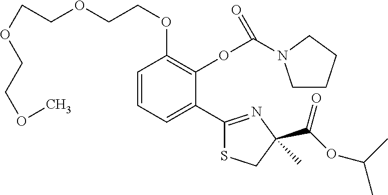

Into a 50-mL 3-necked bottom flask purged and maintained with an inert atmosphere of nitrogen, was placed a solution of (S)-isopropyl 2-(2-hydroxy-3-(2-(2-(2-methoxyethoxy)ethoxy)ethoxy)phenyl)-4-methyl-4,5-dihydrothiazole-4-carboxylate (230 mg, 0.52 mmol, 1.00 equiv) in dichloromethane/Pyridine (4.6/4.6 mL), pyrrolidine-1-carbonyl chloride (905 mg, 6.80 mmol, 13.00 equiv), triethylamine (157.86 mg, 1.56 mmol, 3.00 equiv), 4-dimethylaminopyridine (Cat.4 mg, 0.01 equiv). The resulting solution was stirred for 3 h at room temperature in an oil bath. The resulting mixture was concentrated under vacuum. The crude product (300 mg) was purified by Prep-HPLC with the following conditions (AGILENT Pre-HPLC (UV-Directed)): Column, SunFire Prep C18, 5 um, 19*100 mm; mobile phase, WATER WITH 0.05% TFA and CH3CN (45% CH3CN up to 65% in 7 min, up to 100% in 0.1 min, hold 100% in 0.9 min); Detector, UV 220&254 nm. 240 mg product was obtained. This resulted in 240 mg (85%) of (S)-isopropyl 2-(3-(2-(2-(2-methoxyethoxy)ethoxy)ethoxy)-2-(pyrrolidine-1-carbonyloxy)phenyl)-4-methyl-4,5-dihydrothiazole-4-carboxylate as yellow oil. LC-MS: (ES, m/z): 539 [M+H]⁺. ¹HNMR (CDCl₃, 300 MHz, ppm): δ7.58 (d, J=7.2 Hz, 1H), 7.21 (t, J=8.1 Hz, 1H), 7.10 (d, J=5.1 Hz, 1H), 5.10 (m, 1H), 4.20 (m, 2H), 3.85 (m, 3H), 3.75˜3.45 (m, 12H), 3.40 (s, 3H), 3.26 (d, J=11.4 Hz, 1H), 1.97 (m, 4H), 1.66 (s, 3H), 1.30 (m, 6H).

### EXAMPLE 3

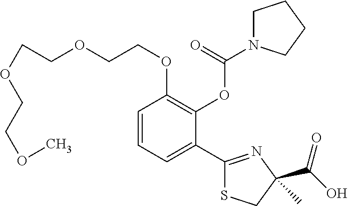

Into a 50-mL round-bottom flask, was placed a solution of (S)-isopropyl 2-(3-(2-(2-(2-methoxyethoxy)ethoxy)ethoxy)-2-(pyrrolidine-1-carbonyloxy)phenyl)-4-methyl-4,5-dihydrothiazole-4-carboxylate (100 mg, 0.18 mmol, 1.00 equiv, 95%) in methanol (20 mL), sodium hydroxide (0.4 mL, 4.00 equiv, 2N). The resulting solution was stirred for 2 h at 20° C. in an oil bath. The pH value of the solution was adjusted to 7 with acetic acid/methanol. The resulting mixture was concentrated under vacuum. The residue was applied onto a silica gel column with dichloromethane/methanol (30:1˜10:1). This resulted in 60 mg (66%) of (S)-2-(3-(2-(2-(2-methoxyethoxy)ethoxy)ethoxy)-2-(pyrrolidine-1-carbonyloxy)phenyl)-4-methyl-4,5-dihydrothiazole-4-carboxylic acid as light yellow oil. LC-MS: (ES, m/z): 497 [M+H]⁺. ¹HNMR (CDCl₃, 400 MHz, ppm): δ 7.43 (d, J=8 Hz, 1H), 7.20 (t, J=8 Hz, 1H), 7.11 (d, J=8 Hz, 1H), 4.18 (m, 2H), 3.83 (m, 2H), 3.71˜3.29 (m, 18H), 1.98 (m, 4H).

### EXAMPLE 4

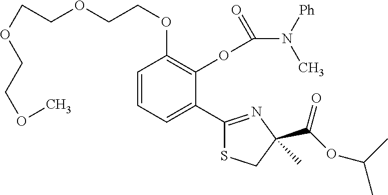

Into a 50-mL 3-necked bottom flask purged and maintained with an inert atmosphere of nitrogen, was placed a solution of (S)-isopropyl 2-(2-hydroxy-3-(2-(2-(2-methoxyethoxy)ethoxy)ethoxy)phenyl)-4-methyl-4,5-dihydrothiazole-4-carboxylate (230 mg, 0.52 mmol, 1.00 equiv) in dichloromethane/Pyridine (4.6/4.6 mL), methyl(phenyl)carbamic chloride (1.06 g, 6.25 mmol, 12.00 equiv), triethylamine (157.86 mg, 1.56 mmol, 3.00 equiv), 4-dimethylaminopyridine (CAT0.4 mg, 0.01 equiv). The resulting solution was stirred for 3 h at room temperature in an oil bath. The resulting mixture was concentrated under vacuum. The crude product (280 mg) was purified by Prep-HPLC with the following conditions (AGILENT Pre-HPLC (UV-Directed)): Column, SunFire Prep C18, 5 um, 19*100 mm; mobile phase, WATER WITH 0.05% TFA and CH₃CN (50% CH3CN up to 70% in 7 min, up to 100% in 0.1 min, hold 100% in 0.9 min); Detector, UV 220&254 nm. 220 mg products were obtained. This resulted in 220 mg (73%) of (S)-isopropyl 2-(3-(2-(2-(2-methoxyethoxy)ethoxy)ethoxy)-2-(methyl(phenyl)carbamoyloxy)phenyl)-4-methyl-4,5-dihydrothiazole-4-carboxylate as yellow oil. LC-MS: (ES, m/z): 575 [M+H]⁺. ¹HNMR (CDCl₃, 300 MHz, ppm): δ 7.7˜07.05 (m, 8H), 5.10 (m, 1H), 4.20 (m, 2H), 3.85 (m, 3H), 3.75˜3.45 (m, 12H), 3.40 (s, 3H), 3.26 (d, J=11.4 Hz, 1H), 1.66 (s, 3H), 1.30 (m, 6H).

### EXAMPLE 5

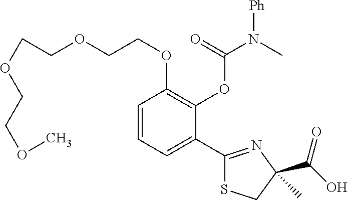

Into a 50-mL round-bottom flask, was placed a solution of (S)-isopropyl 2-(3-(2-(2-(2-methoxyethoxy)ethoxy)ethoxy)-2-(methyl(phenyl)carbamoyloxy)phenyl)-4-methyl-4,5-dihydrothiazole-4-carboxylate (100 mg, 0.17 mmol, 1.00 equiv, 95%) in methanol (20 mL), sodium hydroxide (0.4 mL, 4.00 equiv, 2N). The resulting solution was stirred for 2 h at 20° C. in an oil bath. The pH value of the solution was adjusted to 7 with acetic acid/methanol. The resulting mixture was concentrated under vacuum. The residue was applied onto a silica gel column with dichloromethane/methanol (30:1˜10:1). This resulted in 60 mg (65%) of (S)-2-(3-(2-(2-(2-methoxyethoxy)ethoxy)ethoxy)-2-(methyl(phenyl)carbamoyloxy)phenyl)-4-methyl-4,5-dihydrothiazole-4-carboxylic acid as light yellow oil. LC-MS: (ES, m/z): 533 [M+H]⁺. ¹HNMR (CDCl₃, 400 MHz, ppm): δ 7.5˜47.38 (m, 5H), 7.19˜7.10 (m, 2H), 4.21 (m, 2H), 3.89 (s, 2H), 3.73˜3.28 (m, 15H).

### EXAMPLE 6

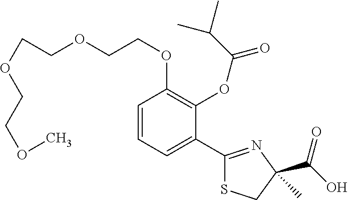

Into a 50-mL 3-necked round-bottom flask purged and maintained with an inert atmosphere of nitrogen, was placed a solution of (S)-2-(2-hydroxy-3-(2-(2-(2-methoxyethoxy)ethoxy)ethoxy)phenyl)-4-methyl-4,5-dihydrothiazole-4-carboxylic acid (200 mg, 0.48 mmol, 1.00 equiv, 95%) in pyridine (20 mL), N,N-dimethylpyridin-4-amine (13 mg, 0.11 mmol, 0.22 equiv, 99%), Isobutyric anhydride (790 mg, 4.94 mmol, 10.39 equiv, 99%). The resulting solution was stirred for 2 days at 25° C. in an oil bath. The resulting mixture was concentrated under vacuum. The residue was applied onto a silica gel column with dichloromethane:methanol (30:1˜10:1). This resulted in 35 mg (15%) of (S)-2-(2-(isobutyryloxy)-3-(2-(2-(2-methoxyethoxy)ethoxy)ethoxy)phenyl)-4-methyl-4,5-dihydrothiazole-4-carboxylic acid as light yellow oil. LC-MS: (ES, m/z): 470 [M+H]⁺. ¹HNMR (CD₃OD, 300 MHz, ppm): δ 7.41˜7.30 (m, 3H), 4.18 (m, 2H), 3.97 (d, J=11.4 Hz, 1H), 3.8 (m, 2H), 3.62 (m, 6H), 3.55 (m, 2H), 3.40 (d, J=11.4 Hz, 1H), 3.36 (m, 3H), 2.92 (m, 1H), 1.65 (s, 3H), 1.35 (m, 6H).

The invention is further illustrated by the following examples, which may not yet have been made or tested.

### EXAMPLE 7

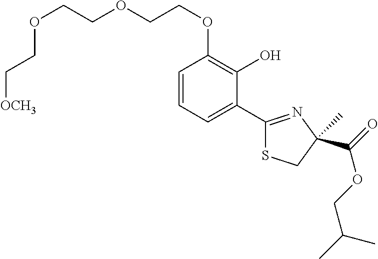

### EXAMPLE 8

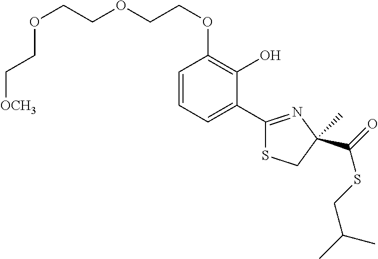

### EXAMPLE 9

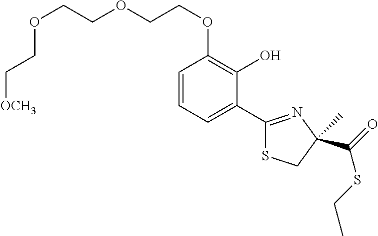

### EXAMPLE 10

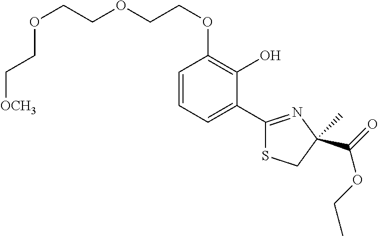

The following compounds can generally be made using the methods known in the art and described above. It is expected that these compounds when made will have activity similar to those that have been made in the examples above.

The invention is further illustrated by the following examples. The following compounds may be represented herein using the Simplified Molecular Input Line Entry System, or SMILES. SMILES is a modern chemical notation system, developed by David Weininger and Daylight Chemical Information Systems, Inc., that is built into all major commercial chemical structure drawing software packages. Software is not needed to interpret SMILES text strings, and an explanation of how to translate SMILES into structures can be found in Weininger, D., J. Chem. Inf. Comput. Sci. 1988, 28, 31-36. All SMILES strings used herein, as well as many IUPAC names, were generated using CambridgeSoft's ChemDraw 11.0.

### OC([C@@]1(C)CSC(C2=CC═C(OCCOCCOCCOC)C═C2OC(C3CCCC3)=O)═N1)=O

### OC([C@@]1(C)CSC(C2=CC═C(OCCOCCOCCOC)C═C2OC(C3=CC═C(OC(F)(F)F) C═C3)=O)═N1)=O

### OC([C@@]1(C)CSC(C2=CC═C(OCCOCCOCCOC)C═C2OC(C3=CC═CC═C3)=O)═N1)=O

### OC([C@@]1(C)CSC(C2=CC═C(OCCOCCOCCOC)C═C2OC(CCC3CCCCC3)=O)═N1)=O

### OC([C@@]1(C)CSC(C2=CC═C(OCCOCCOCCOC)C═C2OC(C3=CC═C(C(F)(F)F)C═C3)=O)═N1)=O

### OC([C@@]1(C)CSC(C2=CC═C(OCCOCCOCCOC)C═C2OC(C3=CC(Cl)═C(OC(F)(F)F)C(Cl)═C3)=O)═N1)=O

### OC([C@@]1(C)CSC(C2=CC═C(OCCOCCOCCOC)C═C2OC(CCCC)═O)═N1)=O

### OC([C@@]1(C)CSC(C2=CC═C(OCCOCCOCCOC)C═C2OC(C3=CC═C(F)C═C3)=O)═N1)=O

### OC([C@@]1(C)CSC(C2=CC═C(OCCOCCOCCOC)C═C2OC(C3=CC(C)═CC(C)═C3)=O)═N1)=O

### OC([C@@]1(C)CSC(C2=CC═C(OCCOCCOCCOC)C═C2OC(CCCCCC)═O)═N1)=O

### OC([C@@]1(C)CSC(C2=CC═C(OCCOCCOCCOC)C═C2OC(CC3=CC(C#N)═CC═C 3F)═O)═N1)=O

### OC([C@@]1(C)CSC(C2=CC═C(OCCOCCOCCOC)C═C2OC(CC3=CC(C(F)(F)F)═C (Cl)C═C3)=O)═N1)=O

### OC([C@@]1(C)CSC(C2=CC═C(OCCOCCOCCOC)C═C2OC(CCC)═O)═N1)=O

### OC([C@@]1(C)CSC(C2=CC═C(OCCOCCOCCOC)C═C2OC(CC3=CC═CC═C3)=O)═N1)=O

### OC([C@@]1(C)CSC(C2=CC═C(OCCOCCOCCOC)C═C2OC(C(C)CCC)═O)═N1)=O

### OC([C@@]1(C)CSC(C2=CC═C(OCCOCCOCCOC)C═C2OC(C(C3=CC═CC═C3)C4=CC═CC═C4)=O)═N1)=O

### OC([C@@]1(C)CSC(C2=CC═C(OCCOCCOCCOC)C═C2OC(N(C)C)═O)═N1)=O

### OC([C@@]1(C)CSC(C2=CC═C(OCCOCCOCCOC)C═C2OC(NC(C)C)═O)═N1)=O

### OC([C@@]1(C)CSC(C2=CC═C(OCCOCCOCCOC)C═C2OC(NCC)═O)═N1)=O

### OC([C@@]1(C)CSC(C2=CC═C(OCCOCCOCCOC)C═C2OC(N(CC)CC)═O)═N1)=O

### OC([C@@]1(C)CSC(C2=CC═C(OCCOCCOCCOC)C═C2OC(N(C)CCC3CCCC3)=O)═N1)=O

### OC([C@@]1(C)CSC(C2=CC═C(OCCOCCOCCOC)C═C2OC(NCC3CC3)=O)═N1)=O

### OC([C@@]1(C)CSC(C2=CC═C(OCCOCCOCCOC)C═C2OC(N3C═CC4=C3C═CC═C4)=O)═N1)=O

### OC([C@@]1(C)CSC(C2=CC═C(OCCOCCOCCOC)C═C2OC(N3C═NC4=C3C═CC═C4)=O)═N1)=O

### OC([C@@]1(C)CSC(C2=CC═C(OCCOCCOCCOC)C═C2OC(N3C═NC═C4C3C═CC═C4)=O)═N1)=O

### OC([C@@]1(C)CSC(C2=CC═C(OCCOCCOCCOC)C═C2OC(N3CCOCC3)=O)═N1)=O

### OC([C@@]1(C)CSC(C2=CC═C(OCCOCCOCCOC)C═C2OC(N3CCN(C)CC3)=O)═N1)=O

### OC([C@@]1(C)CSC(C2=CC═C(OCCOCCOCCOC)C═C2OC(N3CCNCC3)=O)═N1)=O

### OC([C@@]1(C)CSC(C2=CC═C(OCCOCCOCCOC)C═C2OC(C3N═CC═N3)=O)═N1)=O

### OC([C@@]1(C)CSC(C2=CC═C(OCCOCCOCCOC)C═C2OC(N3C═CC═C3)=O)═N1)=O

### OC([C@@]1(C)CSC(C2=CC═C(OCCOCCOCCOC)C═C2OC(N3C═CN═C3)=O)═N1)=O

### OC([C@@]1(C)CSC(C2=CC═C(OCCOCCOCCOC)C═C2OC(NC3=NC═CC═N3)=O)═N1)=O

### OC([C@@]1(C)CSC(C2=CC═C(OCCOCCOCCOC)C═C2OC(C3=CC═NC═N3)=O)═N1)=O

### OC([C@@]1(C)CSC(C2=CC═C(OCCOCCOCCOC)C═C2OC(N3CCSCC3)=O)═N1)=O

### OC([C@@]1(C)CSC(C2=CC═CC(OCCOCCOCCOC)═C2OC(C)═O)═N1)=O

### OC([C@@]1(C)CSC(C2=CC═CC(OCCOCCOCCOC)═C2OC(C3=CC═CC═C3)=O)═N1)=O

### OC([C@@]1(C)CSC(C2=CC═CC(OCCOCCOCCOC)═C2OC(CCC3=CC═CC═C3)=O)═N1)=O

### OC([C@@]1(C)CSC(C2=CC═CC(OCCOCCOCCOC)═C2OC(CCC)═O)═N1)=O

### OC([C@@]1(C)CSC(C2=CC═CC(OCCOCCOCCOC)═C2OC(C3=CC═C(OC)C═C3)=O)═N1)=O

### OC([C@@]1(C)CSC(C2=CC═CC(OCCOCCOCCOC)═C2OC(CC3=C(F)C═CC═C3)=O)═N1)=O

### OC([C@@]1(C)CSC(C2=CC═CC(OCCOCCOCCOC)═C2OC(CCCCC)═O)═N1)=O

### OC([C@@]1(C)CSC(C2=CC═CC(OCCOCCOCCOC)═C2OC(C3CC3)=O)═N1)=O

### OC([C@@]1(C)CSC(C2=CC═CC(OCCOCCOCCOC)═C2OC(C3=C(C)C═C(OC(F)(F)F)C═C3C)═O)═N1)=O

### OC([C@@]1(C)CSC(C2=CC═CC(OCCOCCOCCOC)═C2OC(CC3=CC═CC═C3)=O)═N1)=O

### OC([C@@]1(C)CSC(C2=CC═CC(OCCOCCOCCOC)═C2OC(CCC3CCCC3)=O)═N1)=O

### OC([C@@]1(C)CSC(C2=CC═CC(OCCOCCOCCOC)═C2OC(C3=CC(C(F)(F)F)═C(Cl)C═C3)=O)═N1)=O

### OC([C@@]1(C)CSC(C2=CC═CC(OCCOCCOCCOC)═C2OC(C)═O)═N1)=O

### OC([C@@]1(C)CSC(C2=CC═CC(OCCOCCOCCOC)═C2OC(C3=CC═C(O)C═C3)=O)═N1)=O

### OC([C@@]1(C)CSC(C2=CC═CC(OCCOCCOCCOC)═C2OC(CC3=CC═C(N(C)C)C═C3)=O)═N1)=O

### OC([C@@]1(C)CSC(C2=CC═CC(OCCOCCOCCOC)═C2OC(C)═O)═N1)=O

### OC([C@@]1(C)CSC(C2=CC═CC(OCCOCCOCCOC)═C2OC(C3=CC═C(N)C═C3)=O)═N1)=O

### OC([C@@]1(C)CSC(C2=CC═CC(OCCOCCOCCOC)═C2OC(C(C3=CC═CC═C3)C4=CC═C(C1)C═C4)=O)═N1)=O

### OC([C@@]1(C)CSC(C2=CC═CC(OCCOCCOCCOC)═C2OC(N(C)C)═O)═N1)=O

### OC([C@@]1(C)CSC(C2=CC═CC(OCCOCCOCCOC)═C2OC(NC)═O)═N1)=O

### OC([C@@]1(C)CSC(C2=CC═CC(OCCOCCOCCOC)═C2OC(N3C═CC4=C3C═CC═C4)=O)═N1)=O

### OC([C@@]1(C)CSC(C2=CC═CC(OCCOCCOCCOC)═C2OC(N(CC)CC)═O)═N1)=O

### OC([C@@]1(C)CSC(C2=CC═CC(OCCOCCOCCOC)═C2OC(NCC)═O)═N1)=O

### OC([C@@]1(C)CSC(C2=CC═CC(OCCOCCOCCOC)═C2OC(N3C═NC4=C3C═CC═C4)=O)═N1)=O

### OC([C@@]1(C)CSC(C2=CC═CC(OCCOCCOCCOC)═C2OC(N(CCCN)C)═O)═N1)=O

### OC([C@@]1(C)CSC(C2=CC═CC(OCCOCCOCCOC)═C2OC(NC(C)C)═O)═N1)=O

### OC([C@@]1(C)CSC(C2=CC═CC(OCCOCCOCCOC)═C2OC(N3N═CC4=C3C═CC═C4)=O)═N1)=O

### OC([C@@]1(C)CSC(C2=CC═CC(OCCOCCOCCOC)═C2OC(N(C)CCC)═O)═N1)=O

### OC([C@@]1(C)CSC(C2=CC═CC(OCCOCCOCCOC)═C2OC(NCC(C)(C)C)═O)═N1)=O

### OC([C@@]1(C)CSC(C2=CC═CC(OCCOCCOCCOC)═C2OC(N3CSC4=C3C═CC═C4)=O)═N1)=O

### OC([C@@]1(C)CSC(C2=CC═CC(OCCOCCOCCOC)═C2OC(N(CC3CCCCC3)C)═O)═N1)=O

### OC([C@@]1(C)CSC(C2=CC═CC(OCCOCCOCCOC)═C2OC(NC3CC3)=O)═N1)=O

### OC([C@@]1(C)CSC(C2=CC═CC(OCCOCCOCCOC)═C2OC(N3C═NC═C4C3C═CC═C4)=O)═N1)=O

### OC([C@@]1(C)CSC(C2=CC═CC(OCCOCCOCCOC)═C2OC(N(CC(C)C)C)═O)═N1)=O

### OC([C@@]1(C)CSC(C2=CC═CC(OCCOCCOCCOC)═C2OC(NCC3CCCC3)=O)═N1)=O

### OC([C@@]1(C)CSC(C2=CC═CC(OCCOCCOCCOC)═C2OC(N3C═CC═C4C3C═CC═C4)=O)═N1)=O

### O═C([C@@]1(C)CSC(C2=CC═CC(OCCOCCOCCOC)═C2OC(N3CCCC3)=O)═N1) OCC(C)C

### O═C([C@@]1(C)CSC(C2=CC═CC(OCCOCCOCCOC)═C2OC(N3CCCC3)=O)═N1) SCC(C)C

### O═C([C@@]1(C)CSC(C2=CC═CC(OCCOCCOCCOC)═C2OC(N3CCCC3)=O)═N1) SCC

### O═C([C@@]1(C)CSC(C2=CC═C(OCCOCCOCCOC)C═C2OC(N3CCCC3)=O)═N1) OCC(C)C

### O═C([C@@]1(C)CSC(C2=CC═C(OCCOCCOCCOC)C═C2OC(N3CCCC3)=O)═N1) SCC(C)C

### O═C([C@@]1(C)CSC(C2=CC═C(OCCOCCOCCOC)C═C2OC(N3CCCC3)=O)═N1) SCC

### O═C([C@@]1(C)CSC(C2=CC(OCCOCCOCCOC)═CC═C2OC(N3CCCC3)=O)═N1) OCC(C)C

### O═C([C@@]1(C)CSC(C2=CC(OCCOCCOCCOC)═CC═C2OC(N3CCCC3)=O)═N1) SCC(C)C

### O═C([C@@]1(C)CSC(C2=CC(OCCOCCOCCOC)═CC═C2OC(N3CCCC3)=O)═N1) SCC

### O═C([C@@]1(C)CSC(C2=C(OCCOCCOCCOC)C═CC═C2OC(N3CCCC3)=O)═N1) OCC(C)C

### O═C([C@@]1(C)CSC(C2=C(OCCOCCOCCOC)C═CC═C2OC(N3CCCC3)=O)═N1) SCC(C)C

### O═C([C@@]1(C)CSC(C2=C(OCCOCCOCCOC)C═CC═C2OC(N3CCCC3)=O)═N1) SCC

### O═C([C@@]1(C)CSC(C2=CC═CC(OCCOCCOCCOC)═C2OC(N3CCCCC3)=O)═N1) OCC(C)C

### O═C([C@@]1(C)CSC(C2=CC═CC(OCCOCCOCCOC)═C2OC(N3CCCCC3)=O)═N1) SCC(C)C

### O═C([C@@]1(C)CSC(C2=CC═CC(OCCOCCOCCOC)═C2OC(N3CCCCC3)=O)═N1) SCC

### O═C([C@@]1(C)CSC(C2=CC═C(OCCOCCOCCOC)C═C2OC(N3CCCCC3)=O)═N1) OCC(C)C

### O═C([C@@]1(C)CSC(C2=CC═C(OCCOCCOCCOC)C═C2OC(N3CCCCC3)=O)═N1) SCC(C)C

### O═C([C@@]1(C)CSC(C2=CC═C(OCCOCCOCCOC)C═C2OC(N3CCCCC3)=O)═N1) SCC

### O═C([C@@]1(C)CSC(C2=CC(OCCOCCOCCOC)═CC═C2OC(N3CCCCC3)=O)═N1) OCC(C)C

### O═C([C@@]1(C)CSC(C2=CC(OCCOCCOCCOC)═CC═C2OC(N3CCCCC3)=O)═N1) SCC(C)C

### O═C([C@@]1(C)CSC(C2=CC(OCCOCCOCCOC)═CC═C2OC(N3CCCCC3)=O)═N1) SCC

### O═C([C@@]1(C)CSC(C2=C(OCCOCCOCCOC)C═CC═C2OC(N3CCCCC3)=O)═N1) OCC(C)C

### O═C([C@@]1(C)CSC(C2=C(OCCOCCOCCOC)C═CC═C2OC(N3CCCCC3)=O)═N1) SCC(C)C

### O═C([C@@]1(C)CSC(C2=C(OCCOCCOCCOC)C═CC═C2OC(N3CCCCC3)=O)═N1) SCC

### O═C([C@@]1(C)CSC(C2=CC═CC(OCCOCCOCCOC)═C2OC(CC(C)C)═O)═N1)OC C(C)C

### O═C([C@@]1(C)CSC(C2=CC═CC(OCCOCCOCCOC)═C2OC(CC(C)C)═O)═N1)SC C(C)C

### O═C([C@@]1(C)CSC(C2=CC═CC(OCCOCCOCCOC)═C2OC(CC(C)C)═O)═N1)SC C

### O═C([C@@]1(C)CSC(C2=CC═C(OCCOCCOCCOC)C═C2OC(CC(C)C)═O)═N1)OC C(C)C

### O═C([C@@]1(C)CSC(C2=CC═C(OCCOCCOCCOC)C═C2OC(CC(C)C)═O)═N1)SC C(C)C

### O═C([C@@]1(C)CSC(C2=CC═C(OCCOCCOCCOC)C═C2OC(CC(C)C)═O)═N1)SC C

### O═C([C@@]1(C)CSC(C2=CC(OCCOCCOCCOC)═CC═C2OC(CC(C)C)═O)═N1)OC C(C)C

### O═C([C@@]1(C)CSC(C2=CC(OCCOCCOCCOC)═CC═C2OC(CC(C)C)═O)═N1)SC C(C)C

### O═C([C@@]1(C)CSC(C2=CC(OCCOCCOCCOC)═CC═C2OC(CC(C)C)═O)═N1)SC C

### O═C([C@@]1(C)CSC(C2=C(OCCOCCOCCOC)C═CC═C2OC(CC(C)C)═O)═N1)OC C(C)C

### O═C([C@@]1(C)CSC(C2=C(OCCOCCOCCOC)C═CC═C2OC(CC(C)C)═O)═N1)SC C(C)C

### O═C([C@@]1(C)CSC(C2=C(OCCOCCOCCOC)C═CC═C2OC(CC(C)C)═O)═N1)SC C

### O═C([C@@]1(C)CSC(C2=CC═C(OCCOCCOCCOC)C═C2O)═N1)OCC(C)C

### O═C([C@@]1(C)CSC(C2=CC═C(OCCOCCOCCOC)C═C2O)═N1)SCC(C)C

### O═C([C@@]1(C)CSC(C2=CC═C(OCCOCCOCCOC)C═C2O)═N1)SCC

### O═C([C@@]1(C)CSC(C2=CC(OCCOCCOCCOC)═CC═C2O)═N1)OCC(C)C

### O═C([C@@]1(C)CSC(C2=CC(OCCOCCOCCOC)═CC═C2O)═N1)SCC(C)C

### O═C([C@@]1(C)CSC(C2=CC(OCCOCCOCCOC)═CC═C2O)═N1)SCC

### O═C([C@@]1(C)CSC(C2=C(OCCOCCOCCOC)C═CC═C2O)═N1)OCC(C)C

### O═C([C@@]1(C)CSC(C2=C(OCCOCCOCCOC)C═CC═C2O)═N1)SCC(C)C

### O═C([C@@]1(C)CSC(C2=C(OCCOCCOCCOC)C═CC═C2O)═N1)SCC

### O═C([C@@]1(C)CSC(C2=CC═CC(OCCOCCOCCOC)═C2OC(N(C)C3=CC═CC═C3)=O)═N1)OCC(C)C

### O═C([C@@]1(C)CSC(C2=CC═CC(OCCOCCOCCOC)═C2OC(N(C)C3=CC═CC═C3)=O)═N1)SCC(C)C

### O═C([C@@]1(C)CSC(C2=CC═CC(OCCOCCOCCOC)═C2OC(N(C)C3=CC═CC═C3)=O)═N1)SCC

### O═C([C@@]1(C)CSC(C2=CC═C(OCCOCCOCCOC)C═C2OC(N(C)C3=CC═CC═C3)=O)═N1)OCC(C)C

### O═C([C@@]1(C)CSC(C2=CC═C(OCCOCCOCCOC)C═C2OC(N(C)C3=CC═CC═C3)=O)═N1)SCC(C)C

### O═C([C@@]1(C)CSC(C2=CC═C(OCCOCCOCCOC)C═C2OC(N(C)C3=CC═CC═C3)=O)═N1)SCC

### O═C([C@@]1(C)CSC(C2=CC(OCCOCCOCCOC)═CC═C2OC(N(C)C3=CC═CC═C3)=O)═N1)OCC(C)C

### O═C([C@@]1(C)CSC(C2=CC(OCCOCCOCCOC)═CC═C2OC(N(C)C3=CC═CC═C3)=O)═N1)SCC(C)C

### O═C([C@@]1(C)CSC(C2=CC(OCCOCCOCCOC)═CC═C2OC(N(C)C3=CC═CC═C3)=O)═N1)SCC

### O═C([C@@]1(C)CSC(C2=C(OCCOCCOCCOC)C═CC═C2OC(N(C)C3=CC═CC═C3)=O)═N1)OCC(C)C

### O═C([C@@]1(C)CSC(C2=C(OCCOCCOCCOC)C═CC═C2OC(N(C)C3=CC═CC═C3)=O)═N1)SCC(C)C

### O═C([C@@]1(C)CSC(C2=C(OCCOCCOCCOC)C═CC═C2OC(N(C)C3=CC═CC═C3)=O)═N1)SCC

### O═C([C@@]1(C)CSC(C2=CC═CC(OCCOCCOCCOC)═C2OC(CCC(OCC(C)C)═O)═O)═N1)OCC(C)C

### O═C([C@@]1(C)CSC(C2=CC═CC(OCCOCCOCCOC)═C2OC(CCC(OCC(C)C)═O)═O)═N1)SCC(C)C

### O═C([C@@]1(C)CSC(C2=CC═CC(OCCOCCOCCOC)═C2OC(CCC(OCC(C)C)═O)═O)═N1)SCC

### O═C([C@@]1(C)CSC(C2=CC═C(OCCOCCOCCOC)C═C2OC(CCC(OCC(C)C)═O)═O)═N1)OCC(C)C

### O═C([C@@]1(C)CSC(C2=CC═C(OCCOCCOCCOC)C═C2OC(CCC(OCC(C)C)═O)═O)═N1)SCC(C)C

### O═C([C@@]1(C)CSC(C2=CC═C(OCCOCCOCCOC)C═C2OC(CCC(OCC(C)C)═O)═O)═N1)SCC

### O═C([C@@]1(C)CSC(C2=CC(OCCOCCOCCOC)═CC═C2OC(CCO)═O)═N1)OCC(C)C

### O═C([C@@]1(C)CSC(C2=CC(OCCOCCOCCOC)═CC═C2OC(CCO)═O)═N1)SCC(C)C

### O═C([C@@]1(C)CSC(C2=CC(OCCOCCOCCOC)═CC═C2OC(CCO)═O)═N1)SCC

### O═C([C@@]1(C)CSC(C2=C(OCCOCCOCCOC)C═CC═C2OC(CCO)═O)═N1)OCC(C)C

### O═C([C@@]1(C)CSC(C2=C(OCCOCCOCCOC)C═CC═C2OC(CCO)═O)═N1)SCC(C)C

### O═C([C@@]1(C)CSC(C2=C(OCCOCCOCCOC)C═CC═C2OC(CCO)═O)═N1)SCC

### O═C([C@@]1(C)CSC(C2=CC═CC(OCCOCCOCCOC)═C2OC(N3CCCC3)=O)═N1) OCC4=CC═CC═C4

### O═C([C@@]1(C)CSC(C2=CC═CC(OCCOCCOCCOC)═C2OC(N3CCCC3)=O)═N1) SCC4=CC═CC═C4

### O═C([C@@]1(C)CSC(C2=CC═C(OCCOCCOCCOC)C═C2OC(N3CCCC3)=O)═N1) OCC4=CC═CC═C4

### O═C([C@]1(C)CSC(C2=CC═C(OCCOCCOCCOC)C═C2OC(N3CCCC3)=O)═N1)SC C4=CC═CC═C4

### OC([C@@]1(C)CSC(C2=CC═C(OCCOCCOCCOC)C═C2OC(N3CCCC3)=O)═N1)=O

### O═C([C@@]1(C)CSC(C2=CC(OCCOCCOCCOC)═CC═C2OC(N3CCCC3)=O)═N1) OCC4=CC═CC═C4

### O═C([C@@]1(C)CSC(C2=CC(OCCOCCOCCOC)═CC═C2OC(N3CCCC3)=O)═N1) SCC4=CC═CC═C4

### OC([C@@]1(C)CSC(C2=CC(OCCOCCOCCOC)═CC═C2OC(N3CCCC3)=O)═N1)=O

### O═C([C@@]1(C)CSC(C2=C(OCCOCCOCCOC)C═CC═C2OC(N3CCCC3)=O)═N1) OCC4=CC═CC═C4

### O═C([C@]1(C)CSC(C2=C(OCCOCCOCCOC)C═CC═C2OC(N3CCCC3)=O)═N1)SC C4=CC═CC═C4

### OC([C@@]1(C)CSC(C2=C(OCCOCCOCCOC)C═CC═C2OC(N3CCCC3)=O)═N1)=O

### OC([C@@]1(C)CSC(C2=CC═CC(OCCOCCOCCOC)═C2OC(N3CCCCC3)=O)═N1)=O

### OC([C@@]1(C)CSC(C2=CC═CC(OCCOCCOCCOC)═C2OC(CC(C)C)═O)═N1)=O

### OC([C@@]1(C)CSC(C2=CC═C(OCCOCCOCCOC)C═C2OC(N3CCCCC3)=O)═N1)=O

### OC([C@@]1(C)CSC(C2=CC═C(OCCOCCOCCOC)C═C2OC(CC(C)C)═O)═N1)=O

### OC([C@@]1(C)CSC(C2=CC(OCCOCCOCCOC)═CC═C2OC(N3CCCCC3)=O)═N1)=O

### OC([C@@]1(C)CSC(C2=CC(OCCOCCOCCOC)═CC═C2OC(CC(C)C)═O)═N1)=O

### OC([C@@]1(C)CSC(C2=C(OCCOCCOCCOC)C═CC═C2OC(N3CCCCC3)=O)═N1)=O

### OC([C@@]1(C)CSC(C2=C(OCCOCCOCCOC)C═CC═C2OC(CC(C)C)═O)═N1)=O

### OC([C@@]1(C)CSC(C2=CC═CC(OCCOCCOCCOC)═C2OC(N(C)C3=CC═CC═C3)=O)═N1)=O

### OC([C@@]1(C)CSC(C2=CC═CC(OCCOCCOCCOC)═C2OC(CCC(OCC(C)C)═O)═O)═N1)=O

### OC([C@@]1(C)CSC(C2=CC═C(OCCOCCOCCOC)C═C2OC(N(C)C3=CC═CC═C3)=O)═N1)=O

### OC([C@@]1(C)CSC(C2=CC═C(OCCOCCOCCOC)C═C2OC(CCC(OCC(C)C)═O)═O)═N1)=O

### OC([C@@]1(C)CSC(C2=CC(OCCOCCOCCOC)═CC═C2OC(N(C)C3=CC═CC═C3)=O)═N1)=O

### OC([C@@]1(C)CSC(C2=CC(OCCOCCOCCOC)═CC═C2OC(CCO)═O)═N1)=O

### OC([C@@]1(C)CSC(C2=C(OCCOCCOCCOC)C═CC═C2OC(N(C)C3=CC═CC═C3)=O)═N1)=O

### OC([C@@]1(C)CSC(C2=C(OCCOCCOCCOC)C═CC═C2OC(CCO)═O)═N1)=O

### O═C([C@@]1(C)CSC(C2=CC═CC(OCCOCCOCCOC)═C2O)═N1)OCC3=CC═CC═C3

### O═C([C@@]1(C)CSC(C2=CC═CC(OCCOCCOCCOC)═C2O)═N1)SCC3=CC═CC═C 3

### O═C([C@@]1(C)CSC(C2=CC═CC(OCCOCCOCCOC)═C2O)═N1)OCC

### O═C([C@@]1(C)CSC(C2=CC═C(OCCOCCOCCOC)C═C2O)═N1)OCC3=CC═CC═C3

### O═C([C@]1(C)CSC(C2=CC═C(OCCOCCOCCOC)C═C2O)═N1)SCC3=CC═CC═C3

### O═C([C@@]1(C)CSC(C2=CC═C(OCCOCCOCCOC)C═C2O)═N1)OCC

### O═C([C@@]1(C)CSC(C2=CC(OCCOCCOCCOC)═CC═C2O)═N1)OCC3=CC═CC═C3

### O═C([C@@]1(C)CSC(C2=CC(OCCOCCOCCOC)═CC═C2O)═N1)SCC3=CC═CC═C3

### O═C([C@@]1(C)CSC(C2=CC(OCCOCCOCCOC)═CC═C2O)═N1)OCC

### O═C([C@@]1(C)CSC(C2=C(OCCOCCOCCOC)C═CC═C2O)═N1)OCC3=CC═CC═C3

### O═C([C@]1(C)CSC(C2=C(OCCOCCOCCOC)C═CC═C2O)═N1)SCC3=CC═CC═C3

### O═C([C@@]1(C)CSC(C2=C(OCCOCCOCCOC)C═CC═C2O)═N1)OCC

### OC([C@@]1(C)CSC(C2=CC═CC(OCCOCCOCCOC)═C2OC(N3CCNCC3)=O)═N1)=O

### OC([C@@]1(C)CSC(C2=CC═CC(OCCOCCOCCOC)═C2OC(N3CCCCN(C)C3)=O)═N1)=O

### OC([C@@]1(C)CSC(C2=CC═CC(OCCOCCOCCOC)═C2OC(N3CCCNC3)=O)═N1)=O

### OC([C@@]1(C)CSC(C2=CC═CC(OCCOCCOCCOC)═C2OC(N3CCN(C)CC3)=O)═N1)=O

### OC([C@@]1(C)CSC(C2=CC═CC(OCCOCCOCCOC)═C2OC(N3CCCNCC3)=O)═N 1)=O

### OC([C@@]1(C)CSC(C2=CC═CC(OCCOCCOCCOC)═C2OC(N3CCCN(C)C3)=O)═N1)=O

### OC([C@@]1(C)CSC(C2=CC═CC(OCCOCCOCCOC)═C2OC(N3CCCCCC3)=O)═N1)=O

### OC([C@@]1(C)CSC(C2=CC═CC(OCCOCCOCCOC)═C2OC(N3CCCN(C)CC3)=O)═N1)=O

### OC([C@@]1(C)CSC(C2=CC═CC(OCCOCCOCCOC)═C2OC(N3CCNC3)=O)═N1)=O

### OC([C@@]1(C)CSC(C2=CC═CC(OCCOCCOCCOC)═C2OC(N3CCCCNC3)=O)═N 1)=O

### OC([C@@]1(C)CSC(C2=CC═CC(OCCOCCOCCOC)═C2OC(N3C═CC═C3)=O)═N1)=O

### OC([C@@]1(C)CSC(C2=CC═CC(OCCOCCOCCOC)═C2OC(N3CCN(C)C3)=O)═N 1)=O

### O═C([C@@]1(C)CSC(C2=CC═CC(OCCOCCOCCOC)═C2O)═N1)OC

### O═C([C@@]1(C)CSC(C2=CC═CC(OCCOCCOCCOC)═C2O)═N1)OCCCC

### O═C([C@@]1(C)CSC(C2=CC═CC(OCCOCCOCCOC)═C2O)═N1)SC

### O═C([C@@]1(C)CSC(C2=CC═CC(OCCOCCOCCOC)═C2O)═N1)OCCC

### O═C([C@@]1(C)CSC(C2=CC═CC(OCCOCCOCCOC)═C2O)═N1)OCCC(C)C

### O═C([C@@]1(C)CSC(C2=CC═CC(OCCOCCOCCOC)═C2O)═N1)SCCC

### O═C([C@@]1(C)CSC(C2=CC═CC(OCCOCCOCCOC)═C2O)═N1)OCC(C)(C)C

### O═C([C@@]1(C)CSC(C2=CC═CC(OCCOCCOCCOC)═C2O)═N1)SCCCC

### O═C([C@@]1(C)CSC(C2=CC═CC(OCCOCCOCCOC)═C2O)═N1)SCC(C)(C)C

### O═C([C@@]1(C)CSC(C2=CC═CC(OCCOCCOCCOC)═C2O)═N1)OC(C)C

### O═C([C@@]1(C)CSC(C2=CC═CC(OCCOCCOCCOC)═C2O)═N1)SCCC(C)C

### O═C([C@@]1(C)CSC(C2=CC═CC(OCCOCCOCCOC)═C2O)═N1)SC(C)C

### OC([C@@]1(C)CSC(C2=CC═CC(OCCOCCOCCOC)═C2OC(N3CCOCC3)=O)═N1)=O

### OC([C@@]1(C)CSC(C2=CC═CC(OCCOCCOCCOC)═C2OC(N3CCC3)=O)═N1)=O

### O═C([C@@]1(C)CSC(C2=CC═CC(OCCOCCOC)═C2O)═N1)OCC

### O═C([C@@]1(C)CSC(C2=CC═CC(OCCOCCOC)═C2O)═N1)OC(C)C

### O═C([C@@]1(C)CSC(C2=CC═CC(OCCOCCOC)═C2O)═N1)OCC(C)C

### O═C([C@@]1(C)CSC(C2=CC═CC(OCCOCCOC)═C2O)═N1)SCC(C)C

### O═C([C@@]1(C)CSC(C2=CC═CC(OCCOCCOC)═C2O)═N1)SCC

### O═C([C@@]1(C)CSC(C2=CC═CC(OCCOC)═C2O)═N1)SCC

### O═C([C@@]1(C)CSC(C2=CC═CC(OCCOC)═C2O)═N1)OCC

### O═C([C@@]1(C)CSC(C2=CC═CC(OCCOC)═C2O)═N1)OC(C)C

### O═C([C@@]1(C)CSC(C2=CC═CC(OCCOC)═C2O)═N1)OCC(C)C

### O═C([C@@]1(C)CSC(C2=CC═CC(OCCOC)═C2O)═N1)SCC(C)C

### COCCOCCOC1=C(OC(N2CCCC2)=O)C(C3=N[C@](C(O)═O)(C)CS3)=CC═C1

### COCCOCCOC1=C(OC(N2CCCC2)=O)C(C3=N[C@](C(OC(C)C)═O)(C)CS3)=CC═C1

### COCCOCCOC1=C(OC(N2CCCC2)=O)C(C3=N[C@](C(OCC(C)C)═O)(C)CS3)=CC═C1

### COCCOCCOC1=C(OC(N2CCCC2)=O)C(C3=N[C@](C(OCC)═O)(C)CS3)=CC═C1

### COCCOC1=C(OC(N2CCCC2)=O)C(C3=N[C@](C(SCC(C)C)═O)(C)CS3)=CC═C1

### COCCOC1=C(OC(N2CCCC2)=O)C(C3=N[C@](C(SCC)═O)(C)CS3)=CC═C1

### COCCOCCOC1=C(OC(N2CCCC2)=O)C(C3=N[C@](C(SCC(C)C)═O)(C)CS3)=CC═C1

### COCCOCCOC1=C(OC(N2CCCC2)=O)C(C3=N[C@](C(SCC)═O)(C)CS3)=CC═C1

### COCCOC1=C(OC(N2CCCC2)=O)C(C3=N[C@](C(O)═O)(C)CS3)=CC═C1

### COCCOC1=C(OC(N2CCCC2)=O)C(C3=N[C@](C(OC(C)C)═O)(C)CS3)=CC═C1

### COCCOC1=C(OC(N2CCCC2)=O)C(C3=N[C@](C(OCC(C)C)═O)(C)CS3)=CC═C1

### COCCOC1=C(OC(N2CCCC2)=O)C(C3=N[C@](C(OCC)═O)(C)CS3)=CC═C1

### COCCOCCOCCOC1=C(OC(N2CCCC2)=O)C(C3=N[C@](C(OCC(C)C)═O)(C)C S3)=CC═C1

### COCCOCCOCCOC1=C(OC(N2CCCC2)=O)C(C3=N[C@](C(OCC)═O)(C)CS3)=CC═C1

### COCCOCCOCCOC1=C(OC(N2CCCC2)=O)C(C3=N[C@](C(SCC(C)C)═O)(C)C S3)=CC═C1

### COCCOCCOCCOC1=C(OC(N2CCCC2)=O)C(C3=N[C@](C(SCC)═O)(C)CS3)=CC═C1

### COCCOCCOCCOC1=C(OC(N2CCOCC2)=O)C(C3=N[C@](C(O)═O)(C)CS3)=CC═C1

### COCCOCCOCCOC1=C(OC(N2CCOCC2)=O)C(C3=N[C@](C(OC(C)C)═O)(C)C S3)=CC═C1

### COCCOCCOCCOC1=C(OC(N2CCOCC2)=O)C(C3=N[C@](C(OCC(C)C)═O)(C)CS 3)=CC═C1

### COCCOCCOCCOC1=C(OC(N2CCOCC2)=O)C(C3=N[C@](C(OCC)═O)(C)CS3)=C C═C1

### COCCOCCOCCOC1=C(OC(N2CCOCC2)=O)C(C3=N[C@](C(SCC(C)C)═O)(C)C S3)=CC═C1

### COCCOCCOCCOC1=C(OC(N2CCOCC2)=O)C(C3=N[C@](C(SCC)═O)(C)CS3)=C C═C1

### COCCOCCOC1=C(OC(N2CCOCC2)=O)C(C3=N[C@](C(O)═O)(C)CS3)=CC═C1

### COCCOCCOC1=C(OC(N2CCOCC2)=O)C(C3=N[C@](C(OC(C)C)═O)(C)CS3)=CC═C1

### COCCOCCOC1=C(OC(N2CCOCC2)=O)C(C3=N[C@](C(OCC(C)C)═O)(C)CS3)=C C═C1

### COCCOCCOC1=C(OC(N2CCOCC2)=O)C(C3=N[C@](C(OCC)═O)(C)CS3)=CC═C1

### COCCOC1=C(OC(N2CCOCC2)=O)C(C3=N[C@](C(SCC(C)C)═O)(C)CS3)=CC═C1

### COCCOC1=C(OC(N2CCOCC2)=O)C(C3=N[C@](C(SCC)═O)(C)CS3)=CC═C1

### COCCOCCOC1=C(OC(N2CCOCC2)=O)C(C3=N[C@](C(SCC(C)C)═O)(C)CS3)=C C═C1

### COCCOCCOC1=C(OC(N2CCOCC2)=O)C(C3=N[C@](C(SCC)═O)(C)CS3)=CC═C1

### COCCOC1=C(OC(N2CCOCC2)=O)C(C3=N[C@](C(O)═O)(C)CS3)=CC═C1

### COCCOC1=C(OC(N2CCOCC2)=O)C(C3=N[C@](C(OC(C)C)═O)(C)CS3)=CC═C1

### COCCOC1=C(OC(N2CCOCC2)=O)C(C3=N[C@](C(OCC(C)C)═O)(C)CS3)=CC═C1

### COCCOC1=C(OC(N2CCOCC2)=O)C(C3=N[C@](C(OCC)═O)(C)CS3)=CC═C1

### COCCOCCOCCOC1=C(OC(N(C2=CC═CC═C2)C)═O)C(C3=N[C@](C(OCC)═O)(C) CS3)=CC═C1

### COCCOCCOCCOC1=C(OC(N(C2=CC═CC═C2)C)═O)C(C3=N[C@](C(OCC(C)C)═O)(C)CS3)=CC═C1

### COCCOCCOCCOC1=C(OC(N(C2=CC═CC═C2)C)═O)C(C3=N[C@](C(SCC)═O)(C) CS3)=CC═C1

### COCCOCCOCCOC1=C(OC(N(C2=CC═CC═C2)C)═O)C(C3=N[C@](C(SCC(C)C)═O)(C)CS3)=CC═C1

### COCCOCCOC1=C(OC(N(C2=CC═CC═C2)C)═O)C(C3=N[C@](C(OC(C)C)═O)(C)C S3)=CC═C1

### COCCOCCOC1=C(OC(N(C2=CC═CC═C2)C)═O)C(C3=N[C@](C(O)═O)(C)CS3)=C C═C1

### COCCOCCOC1=C(OC(N(C2=CC═CC═C2)C)═O)C(C3=N[C@](C(OCC)═O)(C)C S3)=CC═C1

### COCCOCCOC1=C(OC(N(C2=CC═CC═C2)C)═O)C(C3=N[C@](C(OCC(C)C)═O)(C) CS3)=CC═C1

### COCCOCCOC1=C(OC(N(C2=CC═CC═C2)C)═O)C(C3=N[C@](C(SCC)═O)(C)C S3)=CC═C1

### COCCOCCOC1=C(OC(N(C2=CC═CC═C2)C)═O)C(C3=N[C@](C(SCC(C)C)═O)(C) CS3)=CC═C1

### COCCOC1=C(OC(N(C2=CC═CC═C2)C)═O)C(C3=N[C@](C(SCC)═O)(C)CS3)=CC═C1

### COCCOC1=C(OC(N(C2=CC═CC═C2)C)═O)C(C3=N[C@](C(SCC(C)C)═O)(C)C S3)=CC═C1

### COCCOC1=C(OC(N(C2=CC═CC═C2)C)═O)C(C3=N[C@](C(OC(C)C)═O)(C)CS3)=CC═C1

### COCCOC1=C(OC(N(C2=CC═CC═C2)C)═O)C(C3=N[C@](C(O)═O)(C)CS3)=CC═C 1

### COCCOC1=C(OC(N(C2=CC═CC═C2)C)═O)C(C3=N[C@](C(OCC)═O)(C)CS3)=CC═C1

### COCCOC1=C(OC(N(C2=CC═CC═C2)C)═O)C(C3=N[C@](C(OCC(C)C)═O)(C)C S3)=CC═C1

### COCCOCCOCCOC1=C(OC(N(C)C)═O)C(C2=N[C@](C(OC(C)C)═O)(C)CS2)=CC═C1

### COCCOCCOCCOC1=C(OC(N(C)C)═O)C(C2=N[C@](C(O)═O)(C)CS2)=CC═C1

### COCCOCCOCCOC1=C(OC(N(C)C)═O)C(C2=N[C@](C(OCC)═O)(C)CS2)=CC═C1

### COCCOCCOCCOC1=C(OC(N(C)C)═O)C(C2=N[C@](C(OCC(C)C)═O)(C)CS2)=CC═C1

### COCCOCCOCCOC1=C(OC(N(C)C)═O)C(C2=N[C@](C(SCC)═O)(C)CS2)=CC═C1

### COCCOCCOCCOC1=C(OC(N(C)C)═O)C(C2=N[C@](C(SCC(C)C)═O)(C)CS2)=CC═C1

### COCCOCCOC1=C(OC(N(C)C)═O)C(C2=N[C@](C(OC(C)C)═O)(C)CS2)=CC═C1

### COCCOCCOC1=C(OC(N(C)C)═O)C(C2=N[C@](C(O)═O)(C)CS2)=CC═C1

### COCCOCCOC1=C(OC(N(C)C)═O)C(C2=N[C@](C(OCC)═O)(C)CS2)=CC═C1

### COCCOCCOC1=C(OC(N(C)C)═O)C(C2=N[C@](C(OCC(C)C)═O)(C)CS2)=CC═C1

### COCCOCCOC1=C(OC(N(C)C)═O)C(C2=N[C@](C(SCC)═O)(C)CS2)=CC═C1

### COCCOCCOC1=C(OC(N(C)C)═O)C(C2=N[C@](C(SCC(C)C)═O)(C)CS2)=CC═C1

### COCCOC1=C(OC(N(C)C)═O)C(C2=N[C@](C(SCC)═O)(C)CS2)=CC═C1

### COCCOC1=C(OC(N(C)C)═O)C(C2=N[C@](C(SCC(C)C)═O)(C)CS2)=CC═C1

### COCCOC1=C(OC(N(C)C)═O)C(C2=N[C@](C(OC(C)C)═O)(C)CS2)=CC═C1

### COCCOC1=C(OC(N(C)C)═O)C(C2=N[C@](C(O)═O)(C)CS2)=CC═C1

### COCCOC1=C(OC(N(C)C)═O)C(C2=N[C@](C(OCC)═O)(C)CS2)=CC═C1

### COCCOC1=C(OC(N(C)C)═O)C(C2=N[C@](C(OCC(C)C)═O)(C)CS2)=CC═C1

The activity of prodrugs of DADFT polyethers as chelating agents may be illustrated in the following assay(s). The compounds listed above, which have not yet been made and/or tested, are predicted to have activity in these assay(s) as well.

### In Vitro Pharmacokinetic Stability Evaluation

Compounds were tested for metabolic stability in human whole blood. Such testing is commonly undertaken prior to or along with advanced preclinical testing in order to identify compounds with desirable pharmacokinetic properties. Into each of 6 centrifuge tubes was added 2 μL of test compound and 198 μL of human whole blood, taken from normal, healthy volunteers, to achieve a final concentration of 5 μM. Tubes were then incubated at 37° C. at approximately 100 rpm on an orbital shaker. One of the tubes was taken at designated time points including 0, 0.5, 1, 4, 6 and 24 hours. The reaction was stopped by the addition of 4 volumes of cold methanol. Samples were centrifuged at 20,000 rpm for 20 minutes to precipitate protein. A 200 μL aliquot of the supernatant was used for LC/MS/MS analysis for each compound at each time point. All experiments were performed in duplicate. The LC system comprised a Shimadzu liquid chromatograph separation system equipped with degasser DGU-20A3, solvent delivery unit LC-20AD, system controller CBM-20A, column oven CTO-10ASVP and CTC Analytics HTC PAL System. Mass spectrometric analysis was performed using an API 4000 instrument from AB Inc. (Canada) with an ESI interface. The data acquisition and control system were created using Analyst 1.5 software from ABI Inc. All calculations were carried out using Microsoft Excel (2003). Percent compound remaining at each time point was estimated by determining the peak areas from extracted ion chromatograms.

| Compound | Half Life, Hours |
|---|---|
| Reference (Mevinolin) | 4-6 |
| Example 1 | 1-4 |
| Example 2 | >24 |
| Example 3 | 1-4 |
| Example 4 | >24 |
| Example 5 | 0.5-1 |
| Example 6 | 6-24 |

### Iron Clearing Efficiency of Prodrugs of DADFT Polyethers

Cannulation of Bile Duct in Non-Iron-Overloaded Rats. The cannulation has been described previously in Bergeron, R J et al., Blood 1993, 81, 2166-2173 and Bergeron, R J et al., Ann. N.Y Acad. Sci. 1990, 612, 378-393. Bile samples is collected from male Sprague-Dawley rats (400-450 g) at 3 h intervals for 24 h. The urine sample is taken at 24 h. Sample collection and handling are as previously described.

Drug Preparation and Administration. In the iron clearing experiments the rats are given a single 50, 150, or 300 mol/kg dose of the drugs po and/or sc. The compounds are administered as a solution in water, 300 mol/kg dose only or (2) as the monosodium salt of the compound of interest (prepared by addition of the free acid to 1 equivalent of NaOH). The chelators are given to the monkeys po and sc at a dose of 150 μmol/kg. The drugs are prepared as for the rats; 2 is given po and sc as a solution in water.

Calculation of Iron Chelator Efficiency. ICE is calculated by dividing the actual amount of iron cleared by a given compound by the theoretical amount that should be cleared. The theoretical iron outputs of the chelators are generated on the basis of a 2:1 ligand:iron complex. The efficiencies in the rats and monkeys are calculated as set forth in Bergeron, R J et al., J. Med. Chem. 1999, 42, 2432-2440. Data are presented as the mean+the standard error of the mean; p-values are generated via a one-tailed Student's 1-test in which the inequality of variances is assumed; and a p-value of <0.05 is considered significant.

Chelator-Induced fron Clearance and Iron Clearing Efficiency in Non-Iron-Overloaded Rodents Dose Response Studies. Because there is a limited amount of chelatable iron available in an animal at any given time, the iron clearance, and therefore iron-clearing efficiency of a ligand, is saturable. The key to managing this phenomenon can be found in the ferrokinetics and the dose-response properties of the ligand. In this regard, the dose-response along with the corresponding ferrokinetics of each compound given po are evaluated in the non-iron-overloaded, bile duct-cannulated rodent model.

Iron-Clearing Efficiency in Non-Iron-Overloaded Rodents and Iron-Loaded Primates: Oral versus Subcutaneous Administration. A similar protocol is carried out to confirm consistence of results and compare the effects of the compounds across species. Cebus apella monkeys and male Sprague-Dawley rats are used, 3-8 per group.

The Iron-Clearing Efficiency protocols and data above are taken from Bergeron, R J et al., “Design, Synthesis, and Testing of Non-Nephrotoxic Desazadesferrithiocin Polyether Analogues,” J Med. Chem. 2008, 51(13), 3913-23. Additional data pertaining to tissue distribution, toxicity, and pharmacokinetics can be found in this publication. Prodrugs of Formula I are expected to show efficacy in this assay.

### Prodrugs of DADFT Polyethers as Lanthanide and Actinide Chelating Agents

The protocol employed in Rao L, Choppin G R, and Bergeron R J, Radiochim. Acta. 88, 851-856 (2000) could be used, optionally with adaptations clear to those of skill in the art, to ascertain the activity of compounds according to the present invention as chelators of lanthanides and actinides. Prodrugs of Formula I are expected to show efficacy in this assay.

From the foregoing description, one skilled in the art can easily ascertain the essential characteristics of this invention, and without departing from the spirit and scope thereof, can make various changes and modifications of the invention to adapt it to various usages and conditions. All references, patents or applications, U.S. or foreign, cited in the application are hereby incorporated by reference as if written herein.

What is claimed is:

1. A compound of Formula I: 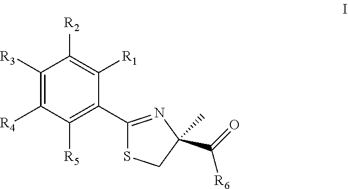
wherein:
R₁, R₂, R₃, R₄, and R₅ are independently chosen from hydrogen, hydroxy, OXR₇, and CH₃O((CH₂)ₙ—O)ₘ—, any of which may be optionally substituted;
m is an integer from 0 to 8;
n is an integer from 0 to 8;
R₆ is chosen from OR₈ and SR₉;
R₇ is chosen from hydrogen, NR₁₀R₁₁, lower alkyl, aralkyl, and aryl, any of which may be optionally substituted;
R₈ is chosen from hydrogen, C₄-C₈ alkyl, and lower aralkyl;
R₉ is chosen from hydrogen, lower alkyl, and lower aralkyl;
R₁₀ and R₁₁ are each independently chosen from hydrogen, lower alkyl, and aryl, any of which may be optionally substituted, or R₁₀ and R₁₁ taken together may form a heterocycloalkyl or heteroaryl; and
X is chosen from a bond and C(O);
wherein at least one of R₁-R₅ is CH₃O((CH₂)ₙ—O)ₘ—;
at least one of R₁-R₅ is optionally substituted OXR₇; and
R₇, R₈, and R₉ can not all be hydrogen.

2. A compound as recited in claim 1 having structural formula II: 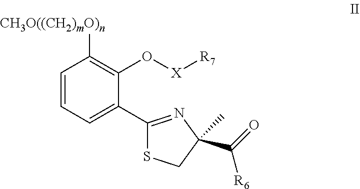
wherein:
m is an integer from 0 to 8;
n is an integer from 0 to 8;
R₆ is chosen from OR₈ and SR₉;
R₇ is chosen from hydrogen, NR₁₀R₁₁, lower alkyl, lower aralkyl, and lower aryl, any of which may be optionally substituted;
R₈ is chosen from hydrogen, C₄-C₈ alkyl, and lower aralkyl;
R₉ is chosen from hydrogen, lower alkyl, and lower aralkyl;
R₁₀ and R₁₁ are each independently chosen from hydrogen, lower alkyl, and aryl, any of which may be optionally substituted, or R₁₀ and R₁₁ taken together may form a lower heterocycloalkyl or lower heteroaryl; and
X is chosen from a bond and C(O);
wherein at least one of R₁-R₅ is CH₃O((CH₂)ₙ—O)ₘ—; and
R₇, R₈, and R₉ can not all be hydrogen.

3. The compound as recited in claim 2, wherein m is 2 and n is 3.

4. The compound as recited in claim 3, wherein
X is C(O); and
R₇ is chosen from NR₁₀R₁₁, lower alkyl, lower aralkyl, and lower aryl, any of which may be optionally substituted.

5. The compound as recited in claim 4, wherein
R₇ is isopropyl.

6. The compound as recited in claim 4, wherein R₇ is NR₁₀R₁₁, wherein R₁₀ and R₁₁ taken together form a lower heterocycloalkyl.

7. The compound as recited in claim 6, wherein R₇ is NR₁₀R₁₁, wherein R₁₀ and R₁₁ taken together form a heterocycloalkyl or heteroaryl chosen from pyrrolidine, piperidine, morpholine, azepine, diazepine, piperazine, or azetidine.

8. The compound as recited in claim 6, wherein
R₈ is chosen from hydrogen, C₄-C₈ alkyl, and lower aralkyl, and
R₉ is chosen from hydrogen, lower alkyl and lower aralkyl.

9. The compound as recited in claim 8, wherein
R₈ is isobutyl; and
R₉ is chosen from ethyl and isobutyl.

10. The compound recited in claim 4, wherein
X is a bond;
R₇ is hydrogen; and
R₈ is chosen from C₄-C₈ alkyl and lower aralkyl;
R₉ is chosen from lower alkyl and lower aralkyl.

11. The compound as recited in claim 10, wherein
R₈ is isobutyl; and
R₉ is chosen from ethyl and isobutyl.

12. The compound as recited in claim 1, having structural formula III: 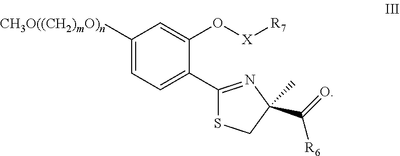
wherein:
m is an integer from 0 to 8;
n is an integer from 0 to 8;
R₆ is chosen from OR₈ and SR₉;
R₇ is chosen from hydrogen, NR₁₀R₁₁, lower alkyl, lower aralkyl, and lower aryl, any of which may be optionally substituted;
R₈ is chosen from hydrogen, C₄-C₈ alkyl, and aralkyl;
R₉ is chosen from hydrogen, alkyl, and aralkyl;
R₁₀ and R₁₁ are each independently chosen from hydrogen, lower alkyl, and aryl, any of which may be optionally substituted, or R₁₀ and R₁₁ taken together may form a lower heterocycloalkyl or heteroaryl; and
X is chosen from a bond and C(O);
wherein at least one of R₁-R₅ is CH₃O((CH₂)ₙ—O)ₘ—; and
R₇, R₈, and R₉ can not all be hydrogen.

13. The compound as recited in claim 12, wherein m is 2 and n is 3.

14. The compound as recited in claim 13, wherein
X is C(O),
R₇ is chosen from NR₁₀R₁₁, lower alkyl, lower aralkyl, and lower aryl, any of which may be optionally substituted.

15. The compound as recited in claim 14, wherein R₇ is NR₁₀R₁₁, wherein R₁₀ and R₁₁ taken together form a lower heterocycloalkyl.

16. The compound as recited in claim 15, wherein R₇ is NR₁₀R₁₁, wherein R₁₀ and R₁₁ taken together form a heterocycloalkyl or heteroaryl selected from the group consisting of pyrrolidine, piperidine, morpholine, azepine, diazepine, piperazine, or azetidine.

17. The compound as recited in claim 15, wherein
R₈ is chosen from hydrogen, C₄-C₈ alkyl, and lower aralkyl; and
R₉ is chosen from hydrogen, lower alkyl and lower aralkyl.

18. The compound as recited in claim 17, wherein
R₈ is isobutyl; and
R₉ is chosen from ethyl and isobutyl.

19. The compound recited in claim 13, wherein
X is a bond;
R₇ is hydrogen;
R₈ is chosen from C₄-C₈ alkyl and lower aralkyl; and
R₉ is chosen from lower alkyl and lower aralkyl.

20. The compound as recited in claim 19, wherein
R₈ is isobutyl; and
R₉ is chosen from ethyl and isobutyl.

21. The compound as recited in claim 1, having structural formula IV: 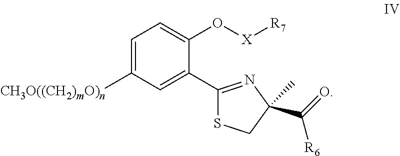
wherein:
m is an integer from 0 to 8;
n is an integer from 0 to 8;
R₆ is chosen from OR₈ and SR₉,
R₇ is chosen from hydrogen, NR₁₀R₁₁, lower alkyl, lower aralkyl, and lower aryl, any of which may be optionally substituted;
R₈ is chosen from hydrogen, C₄-C₈ alkyl, and lower aralkyl;
R₉ is chosen from hydrogen, lower alkyl, and lower aralkyl;
R₁₀ and R₁₁ are each independently chosen from hydrogen, lower alkyl, and aryl, any of which may be optionally substituted, or R₁₀ and R₁₁ taken together may form a lower heterocycloalkyl or heteroaryl; and
X is chosen from a bond and C(O);
wherein at least one of R₁-R₅ is CH₃O((CH₂)ₙ—O)ₘ—; and
R₇, R₈, and R₉ can not all be hydrogen.

22. The compound as recited in claim 21, wherein m is 2 and n is 3.

23. The compound as recited in claim 22, wherein
X is C(O); and
R₇ is chosen from NR₁₀R₁₁, lower alkyl, lower aralkyl, and lower aryl, any of which may be optionally substituted.

24. The compound as recited in claim 23, wherein R₇ is NR₁₀R₁₁, wherein R₁₀ and R₁₁ taken together form a lower heterocycloalkyl.

25. The compound as recited in claim 24, wherein R₇ is NR₁₀R₁₁, wherein R₁₀ and R₁₁ taken together form a heterocycloalkyl or heteroaryl selected from the group consisting of pyrrolidine, piperidine, morpholine, azepine, diazepine, piperazine, or azetidine.

26. The compound as recited in claim 24, wherein
R₈ is chosen from hydrogen, C₄-C₈ alkyl, and lower aralkyl; and
R₉ is chosen from hydrogen, lower alkyl and lower aralkyl.

27. The compound as recited in claim 26, wherein
R₈ is isobutyl; and
R₉ is chosen from ethyl and isobutyl.

28. The compound recited in claim 22, wherein
X is a bond;
R₇ is hydrogen;
R₈ is chosen from C₄-C₈ alkyl and lower aralkyl; and
R₉ is chosen from lower alkyl and lower aralkyl.

29. The compound as recited in claim 28, wherein
R₈ is isobutyl; and
R₉ is chosen from ethyl and isobutyl.

30. The compound as recited in claim 1, having structural formula V: 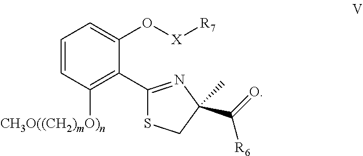
wherein:
m is an integer from 0 to 8;
n is an integer from 0 to 8;
R₆ is chosen from OR₈ and SR₉;
R₇ is chosen from hydrogen, NR₁₀R₁₁, lower alkyl, lower aralkyl, and lower aryl, any of which may be optionally substituted;
R₈ is chosen from hydrogen, C₄-C₈ alkyl, and lower aralkyl;
R₉ is chosen from hydrogen, lower alkyl, and lower aralkyl;
R₁₀ and R₁₁ are each independently chosen from hydrogen, lower alkyl, and aryl, any of which may be optionally substituted, or R₁₀ and R₁₁ taken together may form a lower heterocycloalkyl or heteroaryl; and
X is chosen from a bond and C(O);
wherein at least one of R₁-R₅ is CH₃O((CH₂)ₙ—O)ₘ—; and
R₇, R₈, and R₉ can not all be hydrogen.

31. The compound as recited in claim 30, wherein m is 2 and n is 3.

32. The compound as recited in claim 31, wherein
X is C(O); and
R₇ is chosen from NR₁₀R₁₁, lower alkyl, lower aralkyl, and lower aryl, any of which may be optionally substituted.

33. The compound as recited in claim 32, wherein R₇ is NR₁₀R₁₁, wherein R₁₀ and R₁₁ taken together form a lower heterocycloalkyl.

34. The compound as recited in claim 33, wherein R₇ is NR₁₀R₁₁, wherein R₁₀ and R₁₁ taken together form a heterocycloalkyl or heteroaryl selected from the group consisting of pyrrolidine, piperidine, morpholine, azepine, diazepine, piperazine, or azetidine.

35. The compound as recited in claim 33, wherein
R₈ is chosen from hydrogen, C₄-C₈ alkyl, and lower aralkyl; and
R₉ is chosen from hydrogen, lower alkyl and lower aralkyl.

36. The compound as recited in claim 35, wherein
R₈ is isobutyl; and
R₉ is chosen from ethyl and isobutyl.

37. The compound recited in claim 31, wherein
X is a bond;
R₇ is hydrogen;
R₈ is chosen from C₄-C₈ alkyl and lower aralkyl; and
R₉ is chosen from lower alkyl and lower aralkyl.

38. The compound as recited in claim 37, wherein
R₈ is isobutyl; and
R₉ is chosen from ethyl and isobutyl.

39. A pharmaceutical composition comprising the compound as recited in claim 1, together with at least one pharmaceutically acceptable excipient.

40. A method of treating a metal-mediated condition in a subject comprising administering to the subject a therapeutically effective amount of a compound as recited in claim 1.

41. The method as recited in claim 40 wherein said metal is trivalent.

42. The method as recited in claim 40 wherein said condition is responsive to the chelation, sequestration, or elimination of metal.

43. The method as recited in claim 40 wherein said metal is iron.

44. The method as recited in claim 41 wherein said condition is iron overload.

45. The method as recited in claim 41 wherein said condition is the result of mal-distribution or redistribution of iron in the body.

46. The method as recited in claim 45 wherein said condition is chosen from atransferrinemia, aceruloplasminemia, and Fredreich's ataxia.

47. The method as recited in claim 41 wherein said condition is the result of transfusional iron overload.

48. The method as recited in claim 47 wherein said condition is chosen from beta-thalassemia major and intermedia, sickle cell anemia, Diamond-Blackfan anemia, sideroblastic anemia, chronic hemolytic anemias, off-therapy leukemias, bone marrow transplant and myelodysplastic syndrome.

49. The method as recited in claim 40 wherein said condition is a hereditary condition resulting in the excess absorption of dietary iron.

50. The method as recited in claim 49 wherein said condition is chosen from hereditary hemochromatosis and porphyria cutanea tarda.

51. The method as recited in claim 40 wherein said condition is diabetes.

52. The method as recited in claim 40 wherein said condition is an acquired disease that results in excess dietary iron absorption.

53. The method as recited in claim 52 wherein said condition is a liver disease.

54. The method as recited in claim 53 wherein said disease is hepatitis.

55. The method as recited in claim 40 wherein said metal is a lanthanide or actinide.

56. The method as recited in claim 40 wherein said condition is lanthanide or actinide overload.

57. The method as recited in claim 40 wherein the therapeutically effective amount of a compound thereof as recited in claim 1 that induces the bodily excretion of iron or other trivalent metal is greater than 0.2 mg/kg/d in the subject.

58. The method as recited in claim 40 wherein the therapeutically effective amount of a compound thereof as recited in claim 1 can be given at a dose of at least 10 mg/kg/d without clinically apparent toxic effects on the kidney, bone marrow, thymus, liver, spleen, heart or adrenal glands.
